(19) United States
(12) Patent Application Publication
(10) Pub. No.: US 2008/0039376 A1
(43) Pub. Date: Feb. 14, 2008

(54) Use of Cyclic Anabaenopeptin-type Peptides for the Treatment of a Condition Wherein Inhibition of Carboxypeptidase U is Beneficial, Novel Anabaenopeptin Derivatives and Intermediates Thereof

(75) Inventors: Petter Bjorquist, Goteborg (SE); Malcolm Buchanan, Brisbane (AU); Marc Campitelli, Brisbane (AU); Anthony Carroll, Brisbane (AU); Edward Hyde, Brisbane (AU); Juliette Neve, Brisbane (AU); Ron Quinn, Brisbane (AU); Magnus Polla, Molndal (SE)

Correspondence Address: MORGAN LEWIS & BOCKIUS LLP, 1111 PENNSYLVANIA AVENUE NW, WASHINGTON, DC, 20004, US

(73) Assignee: AZTRAZENECA AB, SE-151 85 SODERTALJE, SWEDEN (SE)

(21) Appl. No.: 10/578,022

(22) PCT Filed: Oct. 28, 2004
(86) PCT No.: PCT/SE04/01568

§ 371 Date: Mar. 6, 2007

(30) Foreign Application Priority Data
Oct. 29, 2003 (SE) 0302853-7

(51) Int. Cl. A61K 38/12; A61P 29/00; A61P 7/00; A61P 7/02; A61P 9/10; C07K 7/50

(52) U.S. Cl. 514/11; 514/9; 530/317

(57) ABSTRACT

The use of a compound of formula (I): in a method of manufacturing a medicament for the treatment or prophylaxis of a condition wherein inhibition of carboxypeptidase U is beneficial; specified compounds of formula (I) and compositions comprising a compound of formula (I) and a pharmaceutically acceptable adjuvant, diluent or carrier.

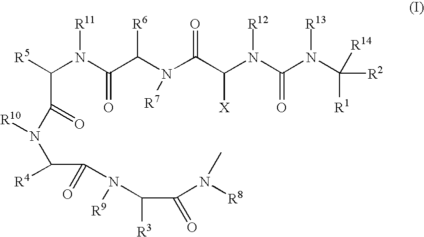

The present invention relates to novel compounds, and pharmaceutically acceptable salts thereof, which inhibit basic carboxypeptidases, more specifically carboxypeptidase U, and thus can be used in the prevention and treatment of diseases wherein inhibition of carboxypeptidase U is beneficial, such as thrombosis and hypercoagulability in blood and tissue, atherosclerosis, adhesions, dermal scarring, cancer, fibrotic conditions, inflammatory diseases and those conditions which benefit from maintaining or enhancing bradykinin levels in the body. In further aspects, the invention relates to compounds of the invention for use in therapy; to processes for preparation of such new compounds; to pharmaceutical compositions containing at least one compound of the invention, or a pharmaceutically acceptable salt thereof, as active ingredient; and to the use of the active compounds in the manufacture of medicaments for the medical use indicated above.

Fibrinolysis is the result of a series of enzymatic reactions resulting in the degradation of fibrin by plasmin. The activation of plasminogen is the central process in fibrinolysis. The cleavage of plasminogen to produce plasmin is accomplished by the plasminogen activators, tissue-type plasminogen activator (t-PA) or urokinase-type plasminogen activator (u-PA). Initial plasmin degradation of fibrin generates carboxy-terminal lysine residues that serve as high affinity binding sites for plasminogen. Since plasminogen bound to fibrin is much more readily activated to plasmin than free plasminogen this mechanism provides a positive feedback regulation of fibrinolysis.

One of the endogenous inhibitors to fibrinolysis is carboxypeptidase U (CPU). CPU is also known as plasma carboxypeptidase B, active thrombin activatable fibrinolysis inhibitor (TAFIa), carboxypeptidase R and inducible carboxypeptidase activity. CPU is formed during coagulation and fibrinolysis from its precursor proCPU by the action of proteolytic enzymes, such as thrombin, thrombin-thrombomodulin complex or plasmin. CPU cleaves basic amino acids at the carboxy-terminal of fibrin fragments. The loss of carboxy-terminal lysines and thereby of lysine binding sites for plasminogen then serves to inhibit fibrinolysis. By inhibiting the loss of lysine binding sites for plasminogen and thus increase the rate of plasmin formation, effective inhibitors of carboxypeptidase U are expected to facilitate fibrinolysis.

2-Mercaptomethyl-3-guanidinoethylthiopropanoic acid is reported as a carboxypeptidase N inhibitor. More recently, this compound has been shown to inhibit CPU, Hendriks, D. et al., Biochimica et Biophysica Acta, 1034 (1990) 86-92.

Guanidinoethylmercaptosuccinic acid is reported as a carboxypeptidase N inhibitor. More recently, this compound has been shown to inhibit CPU, Eaton, D. L., et al., The Journal of Biological Chemistry, 266 (1991) 21833-21838.

CPU inhibitors are disclosed in WO 00/66550, WO 00/66557, WO 03/013526 and WO 03/027128 and a pharmaceutical formulation containing a CPU inhibitor and a thrombin inhibitor is disclosed in WO 00/66152. Inhibitors of plasma carboxypeptidase B are disclosed in WO 01/19836 and WO 03/080631. Inhibitors of TAFIa are disclosed in WO 02/14285, WO 03/061652 and WO 03/061653.

Cyclic Anabaenopeptin-type peptides are disclosed in: Tetrahedron Letters, Vol. 36, No. 9, pp. 1511-1514 (1995); J. Org. Chem. (1997) 62 6199-6203; Tetrahedron Letters, Vol. 36, No. 33, pp. 5933-5936, (1995); J. Nat. Prod. (1996) 59 570-575; Tetrahedron Letters, Vol. 38, No. 31, pp. 5525-5528, (1997); J. Nat. Prod. (1997) 60 139-141; Tetrahedron 54 (1998) 6719-6724; Bioorganic & Medicinal Chemistry Letters 9 (1999) 1243-1246; Tetrahedron 56 (2000) 725-733; J. Nat. Prod. (2000) 63 1280-1282; J. Nat. Prod. (2001) 64 No. 8 1053; Tetrahedron 58 (2002) 6863-6871; and, J. Nat. Prod. (2002) 65 1187-1189.

The synthesis of cyclic Anabaenopeptin-type peptides are disclosed in: Journal of Organic Chemistry, Vol. 62, pp. 6199-6203 (1997); and Angewandte Chemie International Edition, Vol.35, No.12, pp. 1336-1338 (1996). It has now been found that compounds of formula (I):

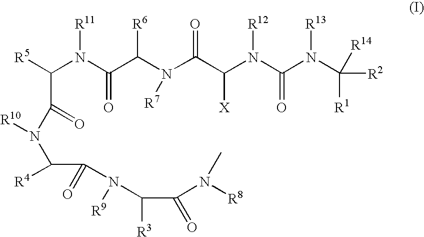

or a pharmaceutically acceptable salt or solvate thereof, or a solvate of such a salt, are particularly effective as inhibitors of carboxypeptidase U and are therefore useful as medicaments for the treatment or prophylaxis of conditions wherein inhibition of carboxypeptidase U is beneficial, for example in the treatment or prophylaxis of: thrombosis and/or hypercoagulability in blood and/or tissues; atherosclerosis; adhesions; dermal scarring; cancer; fibrotic conditions; inflammatory diseases; conditions which benefit from maintaining or enhancing bradykinin levels in the body of a mammal (such as man); protein C resistance; inherited or acquired deficiencies in antithrombin III, protein C, protein S or heparin cofactor II; circulatory or septic shock; circulating antiphospholipid antibodies; hyperhomocysteinemia; heparin induced thrombocytopenia; defects in fibrinolysis; venous thrombosis; pulmonary embolism; arterial thrombosis (for example in myocardial infarction, unstable angina, thrombosis-based stroke or peripheral arterial thrombosis); systemic embolism usually from the atrium during atrial fibrillation or from the left ventricle after transmural myocardial infarction; the prophylaxis of re-occlusion and restenosis (that is, thrombosis) after thrombolysis; percutaneous trans-luminal intervention (PTI) and coronary bypass operations; the prevention of re-thrombosis after microsurgery and vascular surgery in general; disseminated intravascular coagulation caused by bacteria, multiple trauma, intoxication or any other mechanism; fibrinolytic treatment when blood is in contact with foreign surfaces in the body, such as vascular grafts, vascular stents, vascular catheters, mechanical and biological prosthetic valves or any other medical device; fibrinolytic treatment when blood is in contact with medical devices outside the body, such as during cardiovascular surgery using a heart-lung machine or in haemodialysis; prophylaxis of atherosclerotic progression and/or transplant rejection in patients subject to organ transplantation, for example renal transplantation; inhibiting tumor maturation and progression; any condition in which fibrosis is a contributing factor (for example cystic fibrosis, pulmonary fibrotic disease eg chronic obstructive pulmonary disease (COPD), adult respiratory distress syndrome (ARDS), fibromuscular dysplasia, fibrotic lung disease or fibrin deposits in the eye during opthalmic surgery); inflammation (such as asthma, arthritis, endometriosis, inflammatory bowel diseases, psoriasis or atopic dermatitis); neurodegenerative diseases such as Alzheimers and Parkinsons; or conditions known to benefit from maintaining or enhancing bradykinin levels (such as hypertension, angina, heart failure, pulmonary hypertension, renal failure or organ failure).

Thus, the present invention provides the use of a compound of formula (I):

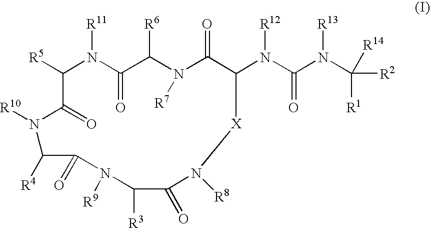

wherein:
X is (CH₂)ₘY(CH₂)ₙ;
m and n are, independently, 1, 2, 3, 4, 5 or 6; provided that m+n is not more than 6;
Y is a bond, O, S(O)ₚ, or S—S;
R¹ is CO₂R¹⁵ or a carboxylic acid isostere such as S(O)₂OH, S(O)₂NHR¹⁵, PO(OR¹⁵)OH, PO(OR¹⁵)NH₂, B(OR¹⁵)₂, PO(R¹⁵)OH, PO(R¹⁵)NH₂ or tetrazole;
R²,R³, R⁴, R⁵ and R⁶ are, independently, hydrogen, C₁₋₆ alkyl (optionally substituted by halogen, hydroxy, cyano, SH, S(O)₃H, S(O)_{q}(C₁₋₆ alkyl), OC(O)(C₁₋₄ alkyl), CF₃, C₁₋₄ alkoxy, OCF₃, COOH, CONH₂, CONH(C₁₋₆ alkyl), NH₂, CNH(NH₂), or NHCNH(NH₂)), C₃₋₆ cycloalkyl(C₁₋₄)alkyl (wherein the cycloalkyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)), heterocyclyl(C₁₋₄)alkyl (wherein the heterocyclyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)), phenyl(C₁₋₄)alkyl (wherein the phenyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)) or heteroaryl(C₁₋₄)alkyl (wherein the heteroaryl ring is optionally substituted by halogen, hydroxy, cyano, C₁₋₄ alkyl, CF₃, C₁₋₄ alkoxy, OCF₃, NH₂, CNH(NH₂) or NHCNH(NH₂));
p and q are, independently, 0, 1 or 2;
R⁷, R⁸, R⁹, R¹⁰, R¹¹, R¹² and R¹³ are, independently, H or C₁₋₄ alkyl;
R¹⁴ is H or C₁₋₄ alkyl; and,
R¹⁵ is H or C₁₋₄ alkyl;
   or a pharmaceutically acceptable salt or solvate thereof, or a solvate of such a salt; in a method of manufacturing a medicament for the treatment or prophylaxis of a condition wherein inhibition of carboxypeptidase U is beneficial, for example in the treatment or prophylaxis of: thrombosis and/or hypercoagulability in blood and/or tissues; atherosclerosis; adhesions; dermal scarring; cancer; fibrotic conditions; inflammatory diseases; conditions which benefit from maintaining or enhancing bradykinin levels in the body of a mammal (such as man); protein C resistance; inherited or acquired deficiencies in antithrombin m, protein C, protein S or heparin cofactor II; circulatory or septic shock; circulating antiphospholipid antibodies; hyperhomocysteinemia; heparin induced thrombocytopenia; defects in fibrinolysis; venous thrombosis; pulmonary embolism; arterial thrombosis (for example in myocardial infarction, unstable angina, thrombosis-based stroke or peripheral arterial thrombosis); systemic embolism usually from the atrium during atrial fibrillation or from the left ventricle after transmural myocardial infarction; the prophylaxis of re-occlusion and restenosis (that is, thrombosis) after thrombolysis; percutaneous trans-luminal intervention (PTI) and coronary bypass operations; the prevention of re-thrombosis after microsurgery and vascular surgery in general; disseminated intravascular coagulation caused by bacteria, multiple trauma, intoxication or any other mechanism; fibrinolytic treatment when blood is in contact with foreign surfaces in the body, such as vascular grafts, vascular stents, vascular catheters, mechanical and biological prosthetic valves or any other medical device; fibrinolytic treatment when blood is in contact with medical devices outside the body, such as during cardiovascular surgery using a heart-lung machine or in haemodialysis; prophylaxis of atherosclerotic progression and/or transplant rejection in patients subject to organ transplantation, for example renal transplantation; inhibiting tumor maturation and progression; any condition in which fibrosis is a contributing factor (for example cystic fibrosis, pulmonary fibrotic disease eg chronic obstructive pulmonary disease (COPD), adult respiratory distress syndrome (ARDS), fibromuscular dysplasia, fibrotic lung disease or fibrin deposits in the eye during opthalmic surgery); inflammation (such as asthma, arthritis, endometriosis, inflammatory bowel diseases, psoriasis or atopic dermatitis); neurodegenerative diseases such as Alzheimers and Parkinsons; or conditions known to benefit from maintaining or enhancing bradykinin levels (such as hypertension, angina, heart failure, pulmonary hypertension, renal failure or organ failure).

In the context of the present invention, the term “therapy” includes “prophylaxis” unless there are specific indications to the contrary. The terms “therapeutic” and “therapeutically” should be understood accordingly.

In one particular aspect the present invention provides the use of a compound of formula (I), as herein described, in a method of manufacturing a medicament for the treatment or prophylaxis of thrombosis and/or hypercoagulability in blood and/or tissues; atherosclerosis; fibrotic conditions; inflammatory diseases; or a condition which benefits from maintaining or enhancing bradykinin levels in the body of a mammal (such as man).

In another aspect the present invention provides the use of a compound of formula (I), as herein described, in a method of manufacturing a medicament for the treatment or prophylaxis of thrombosis and/or hypercoagulability in blood and/or tissues; atherosclerosis; fibrotic conditions; or a condition which benefits from maintaining or enhancing bradykinin levels in the body of a mammal (such as man); for example a medicament for the treatment or prophylaxis of thrombosis and/or hypercoagulability in blood and/or tissues.

The compounds of formula (I) exist in isomeric forms and the present invention covers all such forms and mixtures thereof in all proportions. Both pure enantiomers, racemic mixtures and equal and unequal mixtures of two enantiomers are within the scope of the present invention. It should also be understood that all possible diastereomeric forms possible are within the scope of the invention.

Compounds of formula (I) can be in the form of a salt. Suitable salts include acid addition salts such as a hydrochloride, dihydrochloride, hydrobromide, phosphate, sulfate, acetate, diacetate, fumarate, maleate, tartrate, citrate, oxalate, methanesulfonate or p-toluenesulfonate. Salts also include metal salts, such as an alkali metal salt (for example a sodium or potassium salt) or an alkaline earth metal salt (for example magnesium or calcium).

The term C₁₋₄ alkyl denotes a straight or branched alkyl group having 1 to 4 carbon atoms in the chain. Examples of alkyl include methyl, ethyl, n-propyl, iso-propyl, n-butyl, iso-butyl, sec-butyl and tert-butyl.

The term C₁₋₄ alkoxy denotes an alkyl-O group, where alkyl is straight or branched chain and examples include methoxy and ethoxy.

Halogen includes fluoro, chloro, bromo and iodo (but is, for example, fluoro, chloro or bromo).

Cycloalkyl is, for example, cyclopropyl, cyclopentyl or cyclohexyl.

The term heterocyclyl denotes a non-aromatic ring containing carbon and at least one (such as one or two) atoms selected from nitrogen, oxygen or sulphur. Heterocyclyl is, for example, pyrrolidinyl, piperidinyl, piperazinyl or morpholinyl.

The term heteroaryl denotes an aromatic ring system (for example a mono-cycle or a bicycle) containing carbon and at least one (such as one or two) atoms selected from nitrogen, oxygen or sulphur. Heteroaryl, is for example, furan, thiophene, pyrrole, oxazole, isoxazole, thiazole, imidazole, pyrazole, isothiazole, oxadiazole, furazan, [1,2,3]-triazole, [1,2,4]-triazole, thiadiazole, pyridine, pyridazine, pyrimidine, pyrazine, indole or naphthyridine.

Phenylalkyl is for example benzyl or 1-phenyleth-2-yl.

Cycloalkylalkyl is, for example, cyclohexylmethyl.

Heteroalkylalkyl is, for example, indol-3-ylmethyl.

Heterocyclylalkyl is, for example, piperidin-1-ylmethyl.

In another aspect the present invention provides a compound of formula (I):

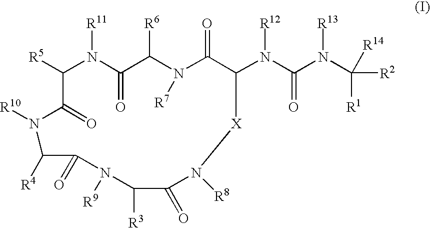

wherein:
X is (CH₂)₄;
R¹ is CO₂R¹⁵;
R² is straight-chain C₁₋₆ alkyl substituted at its terminus by NH₂, CNH(NH₂) or NHCNH(NH₂); C₃₋₆ cycloalkyl substituted by NH₂, CNH(NH₂) or NHCNH(NH₂); heterocyclyl containing at least one nitrogen atom; non-nitrogen containing heterocyclyl substituted with NH₂, CNH(NH₂) or NHCNH(NH₂); heteroaryl substituted with NH₂, CNH(NH₂) or NHCNH(NH₂); phenyl substituted with NH₂, CNH(NH₂) or NHCNH(NH₂); heteroaryl(C₁₋₄)alkyl substituted with NH₂, CNH(NH₂) or NHCNH(NH₂); phenyl(C₁₋₄)alkyl substituted with NH₂, CNH(NH₂) or NHCNH(NH₂); or C₃₋₆ cycloalkyl(C₁₋₄)alkyl substituted with NH₂, CNH(NH₂) or NHCNH(NH₂); all of the above rings being optionally further substituted by one or more of: halogen, hydroxy, cyano, C₁₋₄ alkyl, CF₃, C₁₋₄ alkoxy or OCF₃; one of R³, R⁴, R⁵ and R⁶ is independently, hydrogen, heteroaryl(C₁₋₄)alkyl (wherein the heteroaryl ring is optionally substituted by halogen, hydroxy, cyano, C₁₋₄ alkyl, CF₃, C₁₋₄ alkoxy, OCF₃, NH₂, CNH(NH₂) or NHCNH(NH₂)); and the others are, independently, hydrogen, C₁₋₆ alkyl (optionally substituted by halogen, hydroxy, cyano, SH, S(O)₃H, S(O)_{q}(C₁₋₆ alkyl), OC(O)(C₁₋₄ alkyl), CF₃, C₁₋₄ alkoxy, OCF₃, COOH, CONH₂, CONH(C₁₋₆ alkyl), NH₂, CNH(NH₂), or NHCNH(NH₂)), C₃₋₆ cycloalkyl(C₁₋₄)alkyl (wherein the cycloalkyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)), heterocyclyl(C₁₋₄)alkyl (wherein the heterocyclyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)), phenyl(C₁₋₄)alkyl (wherein the phenyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)) or heteroaryl(C₁₋₄)alkyl (wherein the heteroaryl ring is optionally substituted by halogen, hydroxy, cyano, C₁₋₄ alkyl, CF₃, C₁₋₄ alkoxy, OCF₃, NH₂, CNH(NH₂) or NHCNH(NH₂));
p and q are, independently, 0, 1 or 2;
R⁷, R⁸, R⁹, R¹⁰, R¹¹, R¹² and R¹³ are, independently, H or C₁₋₄ alkyl;
R¹⁴ is H or C₁₋₄ alkyl; and,
R¹⁵ is H or C₁₋₄ alkyl;
   or a pharmaceutically acceptable salt or solvate thereof, or a solvate of such a salt.

In a further aspect the present invention provides a compound of formula (I):

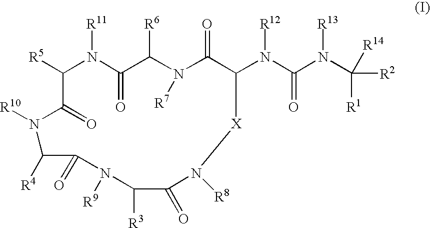

wherein:
R¹ is CO₂R¹⁵;
R² is straight-chain C₁₋₆ alkyl substituted at its terminus by NH₂, CNH(NH₂) or NHCNH(NH₂); C₄ alkyl (such as CH(CH₃)CH₂CH₃ or CH₂CH(CH₃)₂); or (aminopyridinyl)methyl (for example (6-aminopyridin-3-yl)methyl);
one of R³ and R⁴ is (indol-3-yl)CH₂ optionally substituted by halo or hydroxy; and the other is benzyl (optionally substituted by halo or hydroxy) or C₄ alkyl (such as CH(CH₃)CH₂CH₃ or CH₂CH(CH₃)₂);
or R³ and R⁴ are both methyl;
R⁵ and R⁶ are, independently, C₁₋₄ alkyl (for example CH₃, CH(CH₃)₂, CH(CH₃)CH₂CH₃ or CH₂CH(CH₃)₂);
R⁷, R⁸, R⁹, R¹¹, R¹², R¹³ and R¹⁴ are H;
R¹⁰ is C₁₋₄ alkyl; and,
R¹⁵ is H or C₁₋₄ alkyl.

In another aspect the present invention provides a compound of formula (I) having the chirality shown below:

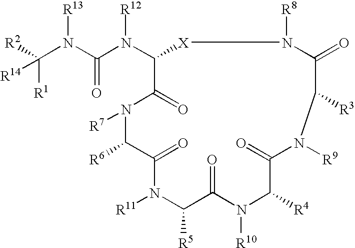

In an aspect of the invention X is (CH₂)₄.

In a further aspect of the invention R¹ is CO₂R¹⁵ wherein R¹⁵ is H or C₁₋₄ alkyl (for example methyl).

In another aspect R² is straight-chain C₁₋₆ alkyl substituted at its terminus by NH₂, CNH(NH₂) or NHCNH(NH₂); C₄ alkyl (such as CH(CH₃)CH₂CH₃ or CH₂CH(CH₃)₂); or (aminopyridinyl)methyl (for example (6-aminopyridin-3-yl)methyl).

In a still further aspect of the invention R² is C₁₋₆ alkyl (such as iso-propyl, CH(CH₃)CH₂CH₃ or CH₂CH(CH₃)₂), benzyl, or straight-chain C₁₋₆ alkyl substituted at its terminus by NH₂, CNH(NH₂), NHCNH(NH₂) or (6-aminopyridin-3-yl)methyl. In another aspect R² is straight-chain C₁₋₆ alkyl substituted at its terminus by NH₂, CNH(NH₂), NHCNH(NH₂) or (6-aminopyridin-3-yl)methyl.

In yet another aspect of the invention R³ is CH₂indolyl (wherein the indolyl is optionally substituted by one or more of: halogen (for example chloro or bromo) or hydroxy), C₁₋₄ alkyl or benzyl (optionally substituted by halogen (for example bromo) or hydroxy).

In another aspect of the invention R⁴ is CH₂indolyl (wherein the indolyl is optionally substituted by one or more of: halogen (for example chloro or bromo) or hydroxy), C₁₋₆ alkyl (such as methyl, iso-propyl, CH(CH₃)CH₂CH₃ or CH₂CH(CH₃)₂) or benzyl (optionally substituted by halogen (for example bromo) or hydroxy).

In a further aspect of the invention R⁵ and R⁶ are, independently, C₁₋₆ alkyl (such as methyl, iso-propyl, CH(CH₃)CH₂CH₃ or CH₂CH(CH₃)₂).

In another aspect of the invention R⁷, R⁸, R⁹, R¹¹, R¹², R¹³ and R¹⁴ are all H.

In yet another aspect of the invention R¹⁰ is C₁₋₄ alkyl (for example methyl).

In a still further aspect the invention provides a compound of formula (I) which is Compound 1, 2, 3, 4, 5, 6, 7, 8, 9, 10, 11, 12, 13, 14, 15 or 16, of a pharmaceutically acceptable salt or solvate thereof, or a solvate of a pharmaceutically acceptable salt thereof.

The compounds of the present invention can be prepared by methods known in the art or analogous to the methods of Examples 3 and 4. It will be appreciated that when adapting methods of the literature or of Examples 3 and 4 that functional groups of intermediate compounds may need to be protected by protecting groups. Functional groups which it is desirable to protect include hydroxy, carboxylate and amino groups. Suitable protecting groups for hydroxy include trialkylsilyl or diarylalkyl-silyl (for example tert-butyldimethylsilyl, tert-butyldiphenylsilyl or trimethylsilyl), tetrahydropyranyl, tert-butyl, methoxymethyl, benzyloxymethyl and 4-methoxybenzyl. Suitable protecting groups for carboxylate include allyl, ethyl, tert-butyl and benzyl esters. Suitable protecting groups for amino include tert-butyloxycarbonyl, 2,4,6-trimethoxybenzyl and benzyloxycarbonyl. The use of protecting groups is described in ‘Protective Groups in Organic Synthesis’, third edition, T. W. Greene & P. G. M. Wutz, Wiley-Interscience (1999). The protective group may also be a polymer resin such as 4-hydroxymethyl-3-methoxyphenoxybutyric acid resin or a 2-chlorotrityl chloride resin.

Thus, compounds of formula I may be prepared by reacting a compound of formula VII

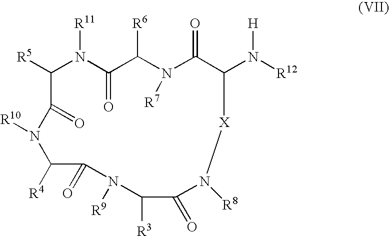

wherein R³ to R¹² and X are as defined above, with a compound of formula VIII

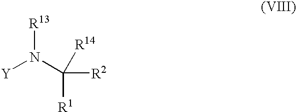

in which R1, R2 R13, R14 are as defined in formula I and Y is an activated acid residue such as 4-nitrophenoxycarbonyl or an activated aminocarbonyl equivalent such as N═C═O. Particular values of Y include activated esters such as 4-nitrophenoxycarbonyl and tert-butoxycarbonyl. A preferred value for Y is 4-nitrophenoxycarbonyl. Other values include those in which YN is an isocyanate group. The reaction will generally be carried out in a suitable solvent such as DMF (or other aprotic solvent) and in the presence of a non-nucleophillic base such as DIEA.

The intermediate of formula VII may be prepared as follows.

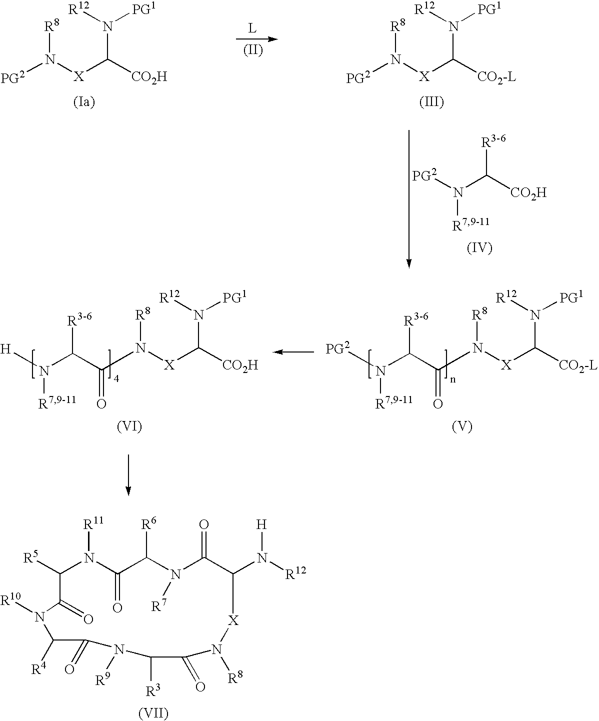

a) Synthesis of Compound III

A compound of formula Ia is dissolved in a nonpolar aprotic solvent such as DCM or THF in the presence of a non-nucleophilic base such as DIEA then reacted with a solid support such as 2-chlorotrityl at room temperature for 2 h. After this time, any unreacted solid support (Compound II) is capped using methanol. The resin is then filtered and washed sequentially with DMF, DCM and DMF.

b) Synthesis of a Compound of Formula (n=4)

A compound of formula III/V (n=1-3) is subjected to solid-phase peptide synthesis as described below:

PG² (in this example Fmoc) is removed from Compound III/V (n=1-3) using 20% piperidine in DMF and the resulting resin washed sequentially with DMF, DCM and DMF. A compound formula IV is preactivated by the addition of a coupling agent such as HBTU or HATU in polar aprotic solvent such as DMF or DMSO, then added to the deprotected the compound of formula III/V (n=1-3). Peptide coupling is initiated by the addition of a non-nucleophilic base such as DIEA and the reaction mixture shaken for 1-2 h. The resin is then filtered and washed sequentially with DMF, DCM and DMF.

b) Synthesis of a Compound of Formula VI

PG² (in this example Fmoc) is removed from Compound V (n=4) using 20% piperidine in DMF and the resulting resin washed sequentially with DMF, DCM and DMF. The compound of formula VI is released from the solid support without the loss of PG¹ by the rapid flow-wash of a compound of formula V (n=4) with dilute acid in aprotic solvent and immediate dilution of the product into a large volume of solvent. A flow wash of 2% TFA in DCM into an equivalent volume of water is an example of this procedure.

b) Synthesis of a Compound of Formula VII

DIEA or equivalent non-nucleophilic base is added to a compound of formula VI in polar aprotic solvent such as DMF or DMSO. The resulting solution of a compound of formula VI is cyclised under conditions of high dilution by dropwise addition to a stirred solution of coupling agent such as PyBOP in polar aprotic solvent such as DMF or DMSO. The reaction mixture is evaporated to dryness and remaining acid-labile protecting groups (eg PG¹) removed using strong acid (TFA, HCl) with added scavengers (TIPS, p-cresol, water or thiocresol). The reaction mixture is again evaporated to dryness before purification by RPHPLC to afford the compound of formula VII. In formula VII PG¹ is a suitable protecting group such as any acid labile nitrogen protecting group, for example, Boc, that is stable to basic conditions required to remove PG². PG² is any base labile nitrogen protecting group such as Fmoc that can be removed without also cleaving the linker L or removing PG¹;

In the above process steps reference to a “coupling agent” refers to any group activating a carboxylic acid towards nucleophilic attack. Examples include precursors to activated esters such as p-nitrophenol and hexafluorophenol, carbodiimide derivatives such as DIC and DCC, benzotriazolyl-tetramethylphosphonium salts such as BOP and PyBOP, benzotriazolyl-tetramethyluronium salts such as HBTU and HATU. L is any extremely acid labile linker for carboxylic acids on solid support that is stable to conditions required to remove PG² such as the 2-chlorotrityl chloride linker, Rink acid resin, 4-hydroxymethyl-3-methoxyphenoxybutyric acid linker.

The novel processes for preparing the intermediates and the novel intermediates referred to herein are also features of the present invention.

Alternatively, a compound of formula (I) can be isolated from natural sources using the methodology of Examples 1 or 2.

The compounds of the invention may also be combined and/or co-administered with any antithrombotic agent with a different mechanism of action, such as an anticoagulant (for example a vitamin K antagonist, an unfractionated or low molecular weight heparin, a synthetic heparin fragment such as fondaparinux, a thrombin inhibitor, a factor Xa inhibitor or other coagulation factor/enzyme inhibitor, a recombinant coagulation factor such as a recombinant human activated protein C) or an antiplatelet agent (such as acetylsalicylic acid, dipyridamole, ticlopidine, clopidogrel or other ADP-receptor [such as a P2Y12 or P2Y1] antagonist, a thromboxane receptor and/or synthetase inhibitor, a fibrinogen receptor antagonist, a prostacyclin mimetic or a phosphodiesterase inhibitor).

The compounds of the invention may further be combined and/or coadministered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction, ischaemic stroke and massive pulmonary embolism.

Thus, in a further aspect the present invention provides a combination (combined and/or co-administered) of a compound of formula (I), wherein X is (CH₂)ₘY(CH₂)ₙ; m and n are, independently, 1, 2, 3, 4, 5 or 6; provided that m+n is not more than 6; Y is a bond, O, S(O)ₚ, or S—S; R¹ is CO₂R¹⁵ or a carboxylic acid isostere such as S(O)₂OH, S(O)₂NHR¹⁵, PO(OR¹⁵)OH, PO(OR¹⁵)NH₂, B(OR¹⁵)₂, PO(R¹⁵)OH, PO(R¹⁵)NH₂ or tetrazole; R², R³, R⁴, R⁵ and R⁶ are, independently, hydrogen, C₁₋₆ alkyl (optionally substituted by halogen, hydroxy, cyano, SH, S(O)₃H, S(O)_{q}(C₁₋₆ alkyl), OC(O)(C₁₋₄ alkyl), CF₃, C₁₋₄ alkoxy, OCF₃, COOH, CONH₂, CONH(C₁₋₆ alkyl), NH₂, CNH(NH₂), or NHCNH(NH₂)), C₃₋₆ cycloalkyl(C₁₋₄)alkyl (wherein the cycloalkyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)), heterocyclyl(C₁₋₄)alkyl (wherein the heterocyclyl ring is optionally substituted by halogen, hydroxy, cyano, C₁₋₄ allyl, CF₃, C₁₋₄ alkoxy, OCF₃, NH₂, CNH(NH₂) or NHCNH(NH₂)), phenyl(C₁₋₄)alkyl (wherein the phenyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)) or heteroaryl(C₁₋₄)alkyl (wherein the heteroaryl ring is optionally substituted by halogen, hydroxy, cyano, C₁₋₄ alkyl, CF₃, C₁₋₄ alkoxy, OCF₃, NH₂, CNH(NH₂) or NHCNH(NH₂)); p and q are, independently, 0, 1 or 2; R⁷, R⁸, R⁹, R¹⁰, R¹¹, R¹² and R¹³ are, independently, H or C₁₋₄ alkyl; R¹⁴ is H or C₁₋₄ alkyl; and, R¹⁵ is H or C₁₋₄ alkyl; or a pharmaceutically acceptable salt or solvate thereof, or a solvate of such a salt; and an antithrombotic agent with a different mechanism of action {such as an anticoagulant (for example a vitamin K antagonist, an unfractionated or low molecular weight heparin, a synthetic heparin fragment such as fondaparinux, a thrombin inhibitor, a factor Xa inhibitor or a recombinant coagulation factor such as a recombinant human activated protein C) or an antiplatelet agent (such as acetylsalicylic acid, dipyridamole, ticlopidine, clopidogrel or other ADP-receptor [such as a P2Y12 or P2Y1] antagonist, a thromboxane receptor and/or synthetase inhibitor, a fibrinogen receptor antagonist, a prostacyclin mimetic or a phosphodiesterase inhibitor)} or a thrombolytic {such as tissue plasminogen activator (natural, recombinant or modified), streptokinase, urokinase, prourokinase, anisoylated plasminogen-streptokinase activator complex (APSAC), animal salivary gland plasminogen activators}.

The compounds of the invention should have a selectivity for carboxypeptidase U over carboxypeptidase N of >50:1, for example >100:1, using the assay described below.

The inhibiting effect of the compounds of the present invention was estimated using the assay described in: Dirk Hendriks, Simon Scharpé and Marc van Sande, Clinical Chemistry, 31, 1936-1939 (1985); and Wei Wang, Dirk F. Hendriks, Simon S. Scharpé, The Journal of Biological Chemistry, 269, 15937-15944 (1994), using a substrate concentration of 4 mM.

The invention also provides a method of treating a condition where inhibition of carboxypeptidase U is beneficial in a mammal suffering from, or at risk of, said condition, which comprises administering to the mammal a therapeutically effective amount of a compound of formula (I), or a pharmaceutically acceptable salt or solvate thereof, or a solvate of such a salt, as hereinbefore defined.

For the above-mentioned therapeutic uses the dosage administered will vary with the compound employed, the mode of administration, the treatment desired and the disorder indicated.

The compounds of formula (I) and pharmaceutically acceptable salts, solvates or solvates of salts thereof may be used on their own but will generally be administered in the form of a pharmaceutical composition in which the formula (I) compound, salt, solvate or solvate of salt (active ingredient) is in association with a pharmaceutically acceptable adjuvant, diluent or carrier. Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

The present invention thus also provides a pharmaceutical composition comprising a compound of formula (I), or a pharmaceutically acceptable salt or solvate thereof, or a solvate of such a salt, as hereinbefore defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt or solvate thereof, or a solvate of such a salt, as hereinbefore defined, with a pharmaceutically acceptable adjuvant, diluent or carrier.

Also included in the invention are derivatives of compounds of formula (I) which have the biological function of compounds of formula (I), such as prodrugs. Prodrugs are, for example, methyl, (pivaloyloxy)methyl esters and [(ethoxycarbonyl)oxy]methyl esters of carboxylic acids.

The following Examples illustrate the invention.

### EXAMPLE 1

This Example describes the isolation of Compounds 1 to 10.

### General Experimental Procedures

Water was Milli-Q filtered, while all other solvents used were Omnisolv. A YMC basic C18 5 uM, 21.2 mm×150 mm, column and Hypersil BDS C18 5 uM, 21.2×150 mm column were used for preparative HPLC. NMR spectra were recorded on a Varian Inova 600 or 500 MHz NMR spectrometer. Samples were dissolved in d₆-DMSO and chemical shifts were calculated relative to the solvent peak (DMSO ¹H □ 2.49 and ¹³C 39.5 ppm). Mass spectra were measured on a Fisons VG Platform II, using positive electrospray ionisation mode. The elution solvent was a mixture acetonitrile/water 50% at 0.1 ml/min.

### Animal Material

The sponge (Melophlus sp.) was collected by SCUBA diving off Ribbon Reef No. 5, Australia and a voucher sample (G319104) is lodged at the Queensland Museum, Brisbane, Australia.

### Extraction and Isolation

A freeze dried ground sample of the sponge Melophlus sp (128 g) collected from Ribbon Reef No. 5 in far North Queensland, Australia was exhaustively extracted with methanol (2 l). The solvent was evaporated to yield a dark brown residue (28 g). The residue was redissolved in a mixture of EtOAc (20 mL) and water (60 mL) and separated by droplet countercurrent chromatography with water as the stationary phase and a gradient from EtOAc to butanol as the mobile phase at 5 mL/min. Two minute fractions were collected and every second fraction analysed by electrospray mass spectrometry. Like fractions were combined yielding 5 fractions. Fraction 2 (320 mg) was separated by centrifugal partition chromatography (Sanki CPC, ascending mode) using a trisolvent mixture CHCl₃/MeOH/H₂O (7:13:8) with the lower phase as stationary phase. A flow rate of 2 mL/min was used and two minute fractions were collected for 360 min. Every second fraction was analyzed by positive electrospray mass spectrometry and like fractions combined. Fractions 91-101 were combined to yield impure Compound 2 (10.8 mg) and fractions 107-120 were combined to yield impure Compound 1 (12.4 mg). The impure peptide fractions of Compounds 1 and 2 were each partitioned between aqueous TFA (1%) and hexane. The aqueous layers from each partition contained pure Compound 2 (9.5 mg) and Compound 1 (11.5 mg). Fractions 1, 3 and 4 from the original DCCC separation were combined with the remaining fractions from the CPC separation and preabsorbed onto C18 (3 g). The preabsorbed fractions were further separated by C18 HPLC hypersil BDS C18 (5 uM, 20 mm×150 mm) using a water/methanol gradient from water containing 1% TFA to methanol containing 1% TFA at 10 mL/min over 60 min. One minute fractions were collected and all fractions analyzed by electrospray mass spectrometry. Like fractions were combined. Fractions 51-58 contained peptides related to Compounds 1 and 2, and were combined (fraction A; 65 mg). This peptide fraction A was further purified by RP HPLC on YMC basic C18 5 uM, 20 mm×150 mm elution with 65% water (containing 1% TFA) and 35% MeCN (containing 1% TFA) at a flow rate of 10 mL/min. Twelve second fractions were collected for 36 minutes. Fractions 58-60 was pure Compound 2 (11 mg), fractions 67-69 was pure Compound 1 (11 mg), fractions 70-72 was pure Compound 3 (2 mg), fractions 73-77 was pure Compound 7 (11.2 mg), fractions 79-82 was pure Compound 4 (7.29 mg), fractions 91-96 was pure Compound 8 (8.75 mg), fractions 101-106 was pure Compound 9 (6.02 mg), fractions 118-125 was pure Compound 5 (2.08 mg), fractions 128-138 was pure Compound 10 (5.73 mg) and fractions 140-150 was pure Compound 6 (5.94 mg).
Compound 1: MS: (positive ESI) [M+H]⁺ m/z 826. ¹H and ¹³C NMR (d₆-DMSO): see Table 1.
Compound 2: MS: (positive ESI) [M+H]⁺ m/z 876, 878. ¹H and ¹³C NMR (d₆-DMSO): see Table 2.
Compound 3: MS: (positive ESI) [M+H]⁺ m/z 890, 892. ¹H and ¹³C NMR (d₆-DMSO): see Table 3.
Compound 4: MS: (positive ESI) [M+H]⁺ m/z 840. ¹H and ¹³C NMR (d₆-DMSO): see Table 4.
Compound 5: MS: (positive ESI) [M+H]⁺ m/z 860, 862. ¹H and ¹³C NMR (d₆-DMSO): see Table 5.
Compound 6: MS: (positive ESI) [M+H]⁺ m/z 861, 863. ¹H and ¹³C NMR (d₆-DMSO): see Table 6.
Compound 7: MS: (positive ESI) [M+H]⁺ m/z 895, 897. ¹H and ¹³C NMR (d₆-DMSO): see Table 7.
Compound 8: MS: (positive ESI) [M+H]⁺ m/z 909, 911. ¹H and ¹³C NMR (d₆-DMSO): see Table 8.
Compound 9: MS: (positive ESI) [M+H]⁺ m/z 909, 911. ¹H and ¹³C NMR (d₆-DMSO): see Table 9.
Compound 10: MS: (positive ESI) [M+H]⁺ m/z 973, 975, 977. ¹H and ¹³C NMR (d₆-DMSO): see Table 10.

After extensive studies including ¹H, gHSQC, gHMBC, and gCOSY experiments, Compounds 1-10 were identified as cyclic peptides. The absolute stereochemistry of Compound 1 was confirmed by single crystal X-ray diffraction analysis.

Compounds 1-5

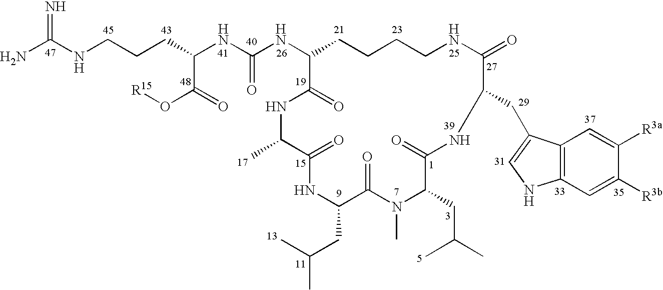

| R^{3a} | R^{3b} | R¹⁵ | |
|---|---|---|---|
| H | H | H | Compound 1 |
| OH | Cl | H | Compound 2 |
| OH | Cl | CH₃ | Compound 3 |
| H | H | CH₃ | Compound 4 |
| H | Cl | H | Compound 5 |

TABLE 1
¹H (600 MHz), ¹³C (125 MHz), HMBC and COSY
NMR data for Compound 1 in d₆-DMSO

| Atom No | ¹³C (mult)^{a} | ¹H (mult, J Hz) | ^{2,3}J_{CH} correlations | COSY |
|---|---|---|---|---|
| N-Methyl leucine | | | | |
| 1 | 169.3 (s) | — | — | — |
| 2 | 58.2 (d) | 4.72 (dd, 5.9, 8.8 Hz, 1H) | 1, 3, 4, 7-NMe, 8 | H3a, H3b |
| 3 | 36.6 (t) | 1.22 (m, 1H) | 1, 2, 5, 6 | H2, H3b, H4 |
| | | 1.63 (m, 1H) | 2, 4, 5, 6 | H2, H3a, H4 |
| 4 | 24.3 (d) | 1.34 (m, 1H) | 2, 3, 5, 6 | H3a, H3b, H5, |
| | | | | H6 |
| 5 | 22.2 (q) | 0.85 (d, 6.8 Hz, 3H) | 3, 4, 6 | H4 |
| 6 | 23.1 (q) | 0.82 (d, 6.8 Hz, 3H) | 3, 4, 5 | H4 |
| NMe | 27.6 (q) | 1.81 (s, 3H) | 2, 8 | — |
| Leucine | | | | |
| 8 | 172.8 (s) | — | — | — |
| 9 | 45.7 (d) | 4.77 (ddd, 2.9, 4.9, 9.8 Hz, 1H) | 10, 11, 8 | H10a, H10b, |
| | | | | H14 |
| 10 | 39.8 (t) | 1.66 (m, 1H) | — | H9, H10b, H11 |
| | | 1.17 (m, 1H) | — | H9, H10a, H11 |
| 11 | 24.7 (d) | 1.82 (m, 1H) | 10 | H10a, H10b, |
| | | | | H12, H13 |
| 12 | 21.6 (q) | 0.87 (d, 6.8 Hz, 3H) | 10, 11, 13 | H11 |
| 13 | 22.9 (q) | 0.91 (d, 6.8 Hz, 3H) | 10, 11, 12 | H11 |
| 14 | — | 8.73 (d, 4.9 Hz, 1H) | 10, 15, 16 | H9 |
| alanine | | | | |
| 15 | 174.1 (s) | — | — | — |
| 16 | 47.9 (d) | 4.20 (dq, 7.8, 7.8 Hz, 1H) | 15, 17 | H17, H18 |
| 17 | 16.7 (q) | 1.30 (d, 7.8 Hz, 3H) | 15, 16 | H16 |
| 18 | — | 7.20 (d, 4.9 Hz, 1H) | 19, 20, 16, 17 | H16 |
| lysine | | | | |
| 19 | 172.7 (s) | — | — | — |
| 20 | 54.6 (d) | 3.92 (ddd, 5.9, 6.8, 6.8 Hz, 1H) | 19, 21, 22, 40 | H21, H26 |
| 21 | 32.5 (t) | 1.65 (m, 2H) | — | H20, H22a, |
| | | | | H22b |
| 22 | 20.3 (t) | 1.40 (m, 1H) | — | H21, H22b, H23 |
| | | 1.10 (m, 1H) | — | H21, H22a, H23 |
| 23 | 28.3 (t) | 1.40 (m, 2H) | — | H22a, H22b, |
| | | | | H24a, H24b |
| 24 | 38.0 (t) | 2.75 (m, 1H) | 27 | H23, H24b, H25 |
| | | 3.58 (m, 1H) | 22, 23 | H23, H24a, H25 |
| 25 | — | 7.44 (dd, 1.2, 7.8 Hz, 1H) | 27 | H24a, H24b |
| 26 | — | 6.45 (d, 6.8 Hz, 1H) | 39, 20, 21 | H20 |
| tryptophan | | | | |
| 27 | 171.4 (s) | — | — | — |
| 28 | 53.9 (d) | 4.40 (ddd, 2.9, 8.8, 11.7 Hz, | 1, 27, 30 | H29a, H29b, |
| | | 1H) | | H39 |
| 29 | 27.9 (t) | 2.88 (dd, 11.7, 13.7 Hz, 1H) | 28, 27, 30, 31, 38 | H28, H29b |
| | | 3.35 (dd, 2.9, 13.7 Hz, 1H) | 28, 27, 30, 31, 38 | H28, H29a |
| 30 | 110.4 (s) | — | — | — |
| 31 | 124.0 (d) | 6.68 (bs, 1H) | 29, 30, 33, 38 | H32 |
| 32 | — | 10.80 (bs, 1H) | 30, 31, 33, 38 | H31 |
| 33 | 136.5 (s) | — | — | — |
| 34 | 111.5 (d) | 7.24 (d, 7.8 Hz, 1H) | 36, 38 | H35, H36 |
| 35 | 121.0 (d) | 7.00 (dd, 7.8, 7.8 Hz, 1H) | 33, 37 | H34, H36 |
| 36 | 118.5 (d) | 6.92 (dd, 7.8, 7.8 Hz, 1H) | 34, 38 | H35, H37 |
| 37 | 116.9 (d) | 7.20 (d, 7.8 Hz, 1H) | 35, 33 | H36, H35 |
| 38 | 127.0 (s) | — | — | — |
| 39 | | 8.62 (d, 8.8 Hz, 1H) | 1, 28, 29 | H28 |
| 40 | 157.5 (s) | — | — | — |
| arginine | | | | |
| 41 | — | 6.42 (d, 7.8 Hz, 1H) | 43, 42, 48, 40 | H42 |
| 42 | 52.9 (d) | 4.05 (ddd, 5.9, 7.8, 7.8 Hz, 1H) | 41, 43, 44, 48 | H41, H43a, |
| | | | | H43b |
| 43 | 29.1 (t) | 1.52 (m, 1H) | — | H42, H44, H43b |
| | | 1.69 (m, 1H) | — | H42, H43a, H44 |
| 44 | 25.1 (t) | 1.40 (m, 2H) | — | H43a, H43b, |
| | | | | H45 |
| 45 | 40.0 (t) | 3.06 (dt, 5.9, 5.9 Hz, 2H) | 43, 44, 47 | H45, H46 |
| 46 | — | 7.64 (t, 5.9 Hz, 1H) | 45, 47 | H45 |
| 47 | 156.9 (s) | — | — | |
| 48 | 175.1 (s) | — | — | — |

^{a}Chemical shifts determined from 2D heteronuclear experiments

TABLE 2
¹H (600 MHz), ¹³C (125 MHz), HMBC and COSY
NMR data for Compound 2 in d₆-DMSO

| Atom No | ¹³C (mult)^{a} | ¹H (mult, J Hz) | ^{2,3}J_{CH} correlations | COSY |
|---|---|---|---|---|
| N-Methyl leucine | | | | |
| 1 | 169.4 (s) | — | — | — |
| 2 | 58.4 (d) | 4.72 (dd, 5.9, 7.8 Hz, 1H) | 1, 3, 4, 8, 7-NMe | H3a, H3b |
| 3 | 36.5 (t) | 1.22 (m, 1H) | 2, 4, 5, 6 | H2, H3b, H4 |
| | | 1.63 (m, 1H) | 2, 4, 5, 6 | H2, H3a, H4 |
| 4 | 23.8 (d) | 1.32 (m, 1H) | 2, 3, 5, 6 | H3a, H3b, H5, H6 |
| 5 | 22.1 (q) | 0.86 (d, 6.8 Hz, 3H) | 3, 4, 6 | H4 |
| 6 | 22.8 (q) | 0.83 (d, 6.8 Hz, 3H) | 3, 4, 5 | H4 |
| NMe | 27.7 (q) | 1.80 (s, 3H) | 2, 8 | — |
| Leucine | | | | |
| 8 | 172.9 (s) | — | — | — |
| 9 | 47.8 (d) | 4.77 (ddd, 2.9, 4.9, 9.8 Hz, 1H) | — | H10a, H10b, H14 |
| 10 | 39.9 (t) | 1.66 (m, 1H) | — | H9, H10b, H11 |
| | | 1.17 (m, 1H) | — | H9, H10a, H11 |
| 11 | 23.4 (d) | 1.82 (m, 1H) | — | H10a, H10b, H12, |
| | | | | H13 |
| 12 | 22.5 (q) | 0.88 (d, 6.8 Hz, 3H) | 10, 11, 13 | H11 |
| 13 | 23.0 (q) | 0.93 (d, 6.8 Hz, 3H) | 10, 11, 12 | H11 |
| 14 | — | 8.74 (d, 5.9 Hz, 1H) | 9, 10, 15 | H9 |
| alanine | | | | |
| 15 | 174.0 (s) | — | — | — |
| 16 | 48.0 (d) | 4.17 (dq, 3.8, 6.8 Hz, 1H) | 15, 17 | H17, H18 |
| 17 | 16.8 (q) | 1.29 (d, 6.8 Hz, 3H) | 15, 16 | H16 |
| 18 | | 7.16 (d, 3.9 Hz, 1H) | 19, 16, 17 | H16 |
| lysine | | | | |
| 19 | 172.5 (s) | — | — | — |
| 20 | 53.9 (d) | 3.92 (ddd, 5.9, 6.8, 6.8 Hz, 1H) | 19, 21, 22, 40 | H21, H26 |
| 21 | 32.9 (t) | 1.57 (m, 2H) | — | H20, H22a, H22b |
| 22 | 20.1 (t) | 1.40 (m, 1H) | — | H21, H22b, H23 |
| | | 1.10 (m, 1H) | — | H21, H22a, H23 |
| 23 | 28.1 (t) | 1.40 (m, 2H) | — | H22a, H22b, H24a, |
| | | | | H24b |
| 24 | 37.0 (t) | 2.75 (m, 1H) | — | H23, H24b, H25 |
| | | 3.56 (m, 1H) | — | H23, H24a, H25 |
| 25 | — | 7.45 (dd, 1.2, 6.8 Hz, 1H) | 27, 19 | H24a, H24b |
| 26 | — | 6.45 (d, 6.8 Hz, 1H) | — | H20 |
| tryptophan | | | | |
| 27 | 170.6 (s) | — | — | — |
| 28 | 53.7 (d) | 4.38 (ddd, 2.9, 8.8, 12.7 Hz, | — | H29a, H29b, H39 |
| | | 1H) | | |
| 29 | 27.9 (t) | 2.83 (dd, 12.7, 12.7 Hz, 1H) | 28, 27, 30, 31, 38 | H28, H29b |
| | | 3.31 (dd, 2.9, 12.7 Hz, 1H) | 28, 27, 30, 31, 38 | H28, H29a |
| 30 | 109.3 (s) | — | — | — |
| 31 | 124.1 (d) | 6.60 (bs, 1H) | 29, 30, 33, 38 | H32 |
| 32 | — | 10.60 (bs, 1H) | 30, 31, 33, 38 | H31 |
| 33 | 131.1 (s) | — | — | — |
| 34 | 111.1 (d) | 7.20 (s, 1H) | 35, 36, 38 | — |
| 35 | 115.0 (s) | — | — | — |
| 36 | 145.9 (s) | — | — | — |
| 37 | 102.1 (d) | 7.01 (s, 1H) | 30, 35, 33, 36 | — |
| 38 | 126.3 (s) | — | — | — |
| 39 | — | 8.64 (d, 9, 8 Hz, 1H) | 1 | H28 |
| 40 | 157.7 (s) | | — | — |
| arginine | | | | |
| 41 | — | 6.36 (d, 5.6 Hz, 1H) | 41, 42, 47 | H42 |
| 42 | 52.7 (d) | 4.07 (ddd. 5.6, 7.8, 7.8 Hz, 1H) | 43, 44, 48 | H41, H43a, H43b |
| 43 | 29.2 (t) | 1.52 (m, 1H) | — | H42, H43b, H44 |
| | | 1.69 (m, 1H) | — | H42, H43a, H44 |
| 44 | 25.3 (t) | 1.46 (m, 2H) | — | H43a, H43b, H45 |
| 45 | 40.7 (t) | 3.06 (m, 2H) | 43, 44, 47 | H46, H45 |
| 46 | — | 7.53 (m, 1H) | 45, 47 | H45 |
| 47 | 157.0 (s) | — | — | — |
| 48 | 174.5 (s) | — | — | — |

^{a}Chemical shifts determined from 2D heteronuclear experiments

TABLE 3
¹H (600 MHz), ¹³C (125 MHz), HMBC and COSY
NMR data for Compound 3 in d₆-DMSO

| Atom No | ¹³C (mult)^{a} | ¹H (mult, J Hz) | ^{2,3}J_{CH} correlations | COSY |
|---|---|---|---|---|
| N-Methyl leucine | | | | |
| 1 | 168.9 (s) | — | — | — |
| 2 | 57.2 (d) | 4.77 (dd, 5.9, 8.8 Hz, 1H) | 8 | H3a, H3b |
| 3 | 35.9 (t) | 1.20 (m, 1H) | — | H2, H3b, H4 |
| | | 1.71 (m, 1H) | — | H2, H3a, H4 |
| 4 | 24.2 (d) | 1.35 (m, 1H) | — | H3a, H3b, H5, H6 |
| 5 | 23.0 (q) | 0.85 (d, 6.8 Hz, 3H) | 3, 4, 6 | H4 |
| 6 | 23.3 (q) | 0.88 (d, 6.8 Hz, 3H) | 3, 4, 5 | H4 |
| NMe | 26.9 (q) | 1.87 (s, 3H) | 2, 8 | — |
| Leucine | | | | |
| 8 | 172.2 (s) | — | — | — |
| 9 | 47.8 (d) | 4.79 (ddd, 2.9, 4.9, 9.8 Hz, 1H) | — | H10a, H10b, H14 |
| 10 | 39.4 (t) | 1.70 (m, 1H) | — | H9, H10b, H11 |
| | | 1.22 (m, 1H) | — | H9, H10a, H11 |
| 11 | 24.1 (d) | 1.84 (m, 1H) | — | H10a, H10b, H12, |
| | | | | H13 |
| 12 | 21.5 (q) | 0.90 (d, 6.8 Hz, 3H) | 10, 11, 13 | H11 |
| 13 | 23.0 (q) | 0.95 (d, 6.8 Hz, 3H) | 10, 11, 12 | H11 |
| 14 | — | 8.76 (d, 4.9 Hz, 1H) | 15 | H9 |
| alanine | | | | |
| 15 | 173.6 (s) | — | — | — |
| 16 | 47.5 (d) | 4.19 (dq, 5.8, 6.8 Hz, 1H) | — | H17, H18 |
| 17 | 16.5 (q) | 1.32 (d, 6.8 Hz, 3H) | 15, 16 | H16 |
| 18 | — | 7.22 (d, 5.9 Hz, 1H) | 19 | H16 |
| lysine | | | | |
| 19 | 171.9 (s) | — | — | — |
| 20 | 54.2 (d) | 3.94 (ddd, 5.9, 6.8, 6.8 Hz, 1H) | 19, 21, 22 | H21, H26 |
| 21 | 31.7 (t) | 1.60 (m, 2H) | — | H20, H22a, H22b |
| 22 | 20.1 (t) | 1.40 (m, 1H) | — | H21, H22b, H23 |
| | | 1.10 (m, 1H) | — | H21, H22a, H23 |
| 23 | 27.2 (t) | 1.40 (m, 2H) | — | H22a, H22b, H24a, |
| | | | | H24b |
| 24 | 38.1 (t) | 2.78 (m, 1H) | 27 | H23, H24b, H25 |
| | | 3.60 (m, 1H) | — | H23, H24a, H25 |
| 25 | — | 7.42 (dd, 1.2, 7.8 Hz, 1H) | 27 | H24a, H24b |
| 26 | — | 6.31 (d, 6.8 Hz, 1H) | 40 | H20 |
| tryptophan | | | | |
| 27 | 172.8 (s) | — | — | — |
| 28 | 53.7 (d) | 4.39 (ddd, 2.9, 8.8, 11.7 Hz, | — | H29a, H29b, H39 |
| | | 1H) | | |
| 29 | 27.9 (t) | 2.86 (dd, 11.7, 13.7 Hz, 1H) | 28, 27, 30, 31, 38 | H28, H29b |
| | | 3.27 (dd, 2.9, 13.7 Hz, 1H) | 28, 27, 30, 31, 38 | H28, H29a |
| 30 | 109.4 (s) | — | — | — |
| 31 | 124.5 (d) | 6.62 (bs, 1H) | 29, 30, 33, 38 | H32 |
| 32 | — | 10.65 (bs, 1H) | 30, 31, 33, 38 | H31 |
| 33 | 130.4 (s) | — | — | — |
| 34 | 111.2 (d) | 7.22 (s, 1H) | 36, 38 | — |
| 35 | 115.3 (s) | — | — | — |
| 36 | 145.6 (s) | — | — | — |
| 37 | 102.5 (d) | 7.00 (s, 1H) | 30, 35, 33 | — |
| 38 | 125.9 (s) | — | — | — |
| 39 | — | 8.67 (d, 8.8 Hz, 1H) | | H28 |
| 40 | 157.2 (s) | — | — | — |
| arginine | | | | |
| 41 | — | 6.50 (d, 7.8 Hz, 1H) | 40 | H42 |
| 42 | 51.9 (d) | 4.05 (ddd, 5.9, 7.8, 7.8 Hz, 1H) | 47 | H41, H43a, H43b |
| 43 | 28.7 (t) | 1.56 (m, 1H) | — | H42, H43b, H44 |
| | | 1.74 (m, 1H) | — | H42, H43a, H44 |
| 44 | 24.9 (t) | 1.46 (m, 2H) | — | H43a, H43b, H45 |
| 45 | 39.7 (t) | 3.09 (dt, 5.9, 5.9 Hz, 2H) | 47 | H46, H45 |
| 46 | — | 7.42 (t, 5.9 Hz, 1H) | 47 | H45 |
| 47 | 156.4 (s) | — | — | — |
| 48 | 173.1 (s) | — | — | — |
| 48-Me | 51.8 (q) | 3.62 (s, 3H) | 48 | — |

^{a}Chemical shifts determined from 2D heteronuclear experiments

TABLE 4
¹H (600 MHz), ¹³C (125 MHz), and COSY
NMR data for Compound 4 in d₆-DMSO

| Atom No | ¹³C (mult)^{a} | ¹H (mult, J Hz) | COSY |
|---|---|---|---|
| N-Methyl leucine | | | |
| 1 | n.o. | — | — |
| 2 | 57.9 (d) | 4.78 (dd, 5.9, 8.8 Hz, 1H) | H3a, H3b |
| 3 | 36.1 (t) | 1.27 (m, 1H) | H2, H3b, H4 |
| | | 1.68 (m, 1H) | H2, H3a, H4 |
| 4 | 24.1 (d) | 1.37 (m, 1H) | H3a, H3b, H5, H6 |
| 5 | 23.7 (q) | 0.79 (d, 6.8 Hz, 3H) | H4 |
| 6 | 20.9 (q) | 0.83 (d, 6.8 Hz, 3H) | H4 |
| NMe | 27.3 (q) | 1.81 (s, 3H) | — |
| Leucine | | | |
| 8 | n.o. | — | — |
| 9 | 47.0 (d) | 4.78 (ddd, 2.9, 4.9, 9.8 Hz, 1H) | H10a, H10b, H14 |
| 10 | 40.0 (t) | 1.63 (m, 1H) | H9, H10b, H11 |
| | | 1.25 (m, 1H) | H9, H10a, H11 |
| 11 | 24.2 (d) | 1.83 (m, 1H) | H10a, H10b, H12, |
| | | | H13 |
| 12 | 20.7 (q) | 0.84 (d, 6.8 Hz, 3H) | H11 |
| 13 | 23.9 (q) | 0.91 (d, 6.8 Hz, 1H) | H11 |
| 14 | — | 8.79 (d, 4.9 Hz, 1H) | H9 |
| alanine | | | |
| 15 | n.o. | — | — |
| 16 | 47.3 (d) | 4.19 (dq, 7.8, 7.8 Hz, 1H) | H17, H18 |
| 17 | 16.2 (q) | 1.33 (d, 7.8 Hz, 3H) | H16 |
| 18 | — | 7.29 (d, 4.9 Hz, 1H) | H16 |
| lysine | | | |
| 19 | n.o. | — | — |
| 20 | 54.3 (d) | 3.87 (ddd, 5.9, 6.8, 6.8 Hz, 1H) | H21, H26 |
| 21 | 32.1 (t) | 1.60 (m, 2H) | H20, H22a, H22b |
| 22 | 21.1 (t) | 1.40 (m, 1H) | H21, H22b, H23 |
| | | 1.10 (m, 1H) | H21, H22a, H23 |
| 23 | 28.1 (t) | 1.40 (m, 2H) | H22a, H22b, H24a, |
| | | | H24b |
| 24 | 38.1 (t) | 2.75 (m, 1H) | H23, H24b, H25 |
| | | 3.59 (m, 1H) | H23, H24a, H25 |
| 25 | — | 7.41 (dd, 1.2, 7.8 Hz, 1H) | H24a, H24b |
| 26 | — | 6.39 (d, 6.8 Hz, 1H) | H20 |
| tryptophan | | | |
| 27 | n.o. | — | — |
| 28 | 53.8 (d) | 4.38 (ddd, 2.9, 8.8, 11.7 Hz, | H29a, H29b, H39 |
| | | 1H) | |
| 29 | 27.6 (t) | 2.81 (dd, 11.7, 13.7 Hz, 1H) | H28, H29b |
| | | 3.37 (dd, 2.9, 13.7 Hz, 1H) | H28, H29a |
| 30 | n.o. | — | — |
| 31 | 124.5 (d) | 6.72 (bs, 1H) | H32 |
| 32 | — | 10.80 (bs, 1H) | H31 |
| 33 | n.o. | — | — |
| 34 | 111.2 (d) | 7.37 (d, 7.8 Hz, 1H) | H35 |
| 35 | 120.2 (d) | 6.89 (dd, 7.8, 7.8 Hz, 1H) | H34, H36 |
| 36 | 121.0 (d) | 7.00 (dd, 7.8, 7.8 Hz, 1H) | H35, H37 |
| 37 | 117.8 (d) | 7.21 (d, 7.8 Hz, 1H) | H36, H35 |
| 38 | n.o. | — | — |
| 39 | — | 8.64 (d, 8.8 Hz, 1H) | H28 |
| 40 | n.o. | — | — |
| arginine | | | |
| 41 | — | 6.49 (d, 7.8 Hz, 1H) | H42 |
| 42 | 52.2 (d) | 4.19 (ddd, 5.9, 7.8, 7.8 Hz, 1H) | H41, H43a, H43b |
| 43 | 28.0 (t) | 1.52 (m, 1H) | H42, H43b, H44 |
| | | 1.71 (m, 1H) | H42, H43a, H44 |
| 44 | 24.7 (t) | 1.40 (m, 2H) | H43a, H43b, H45 |
| 45 | 40.1 (t) | 3.07 (dt, 5.9, 5.9 Hz, 2H) | H46, H45 |
| 46 | — | 7.42 (t, 5.9 Hz, 1H) | H45 |
| 47 | n.o. | | |
| 48 | n.o. | — | — |
| 48-Me | 52.1 (q) | 3.58 (s, 3H) | — |

^{a}Chemical shifts determined from 2D heteronuclear experiments
n.o. = not observed

TABLE 5
¹H (600 MHz), ¹³C (125 MHz), HMBC and COSY
NMR data for Compound 5 in d₆-DMSO

| Atom No | ¹³C (mult)^{a} | ¹H (mult, J Hz) | ^{2,3}J_{CH} correlations | COSY |
|---|---|---|---|---|
| N-Methyl leucine | | | | |
| 1 | 168.9 (s) | — | — | — |
| 2 | 57.5 (d) | 4.76 (dd, 5.9, 8.8 Hz, 1H) | 1, 3, 8, 7-NMe | H3a, H3b |
| 3 | 36.6 (t) | 1.27 (m, 1H) | — | H2, H3b, H4 |
| | | 1.65 (m, 1H) | — | H2, H3a, H4 |
| 4 | 24.4 (d) | 1.34 (m, 1H) | — | H3a, H3b, H5, H6 |
| 5 | 23.7 (q) | 0.82 (d, 6.8 Hz, 3H) | 3, 4, 6 | H4 |
| 6 | 21.2 (q) | 0.84 (d, 6.8 Hz, 3H) | 3, 4, 5 | H4 |
| NMe | 27.5 (q) | 1.77 (s, 3H) | 2, 8 | — |
| Leucine | | | | |
| 8 | 172.6 (s) | — | — | — |
| 9 | 46.8 (d) | 4.77 (ddd, 2.9, 4.9, 9.8 Hz, 1H) | — | H10a, H10b, H14 |
| 10 | 40.0 (t) | 1.68 (m, 1H) | 9, 11 | H9, H10b, H11 |
| | | 1.22 (m, 1H) | — | H9, H10a, H11 |
| 11 | 24.5 (d) | 1.82 (m, 1H) | — | H10a, H10b, H12, |
| | | | | H13 |
| 12 | 21.4 (q) | 0.86 (d, 6.8 Hz, 3H) | 10, 11, 13 | H11 |
| 13 | 23.0 (q) | 0.90 (d, 6.8 Hz, 3H) | 10, 11, 12 | H11 |
| 14 | — | 8.77 (d, 4.9 Hz, 1H) | 9, 10, 15 | H9 |
| alanine | | | | |
| 15 | 173.8 (s) | — | — | — |
| 16 | 48.2 (d) | 4.16 (dq, 4.9, 7.8 Hz, 1H) | 15, 17 | H17, H18 |
| 17 | 16.8 (q) | 1.27 (d, 7.8 Hz, 3H) | 15, 16 | H16 |
| 18 | — | 7.18 (d, 4.9 Hz, 1H) | 19 | H16 |
| lysine | | | | |
| 19 | 172.3 (s) | — | — | — |
| 20 | 54.1 (d) | 3.91 (ddd, 5.9, 6.8, 6.8 Hz, 1H) | 19, 21, 22 | H21, H26 |
| 21 | 32.1 (t) | 1.60 (m, 2H) | — | H20, H22a, H22b |
| 22 | 20.6 (t) | 1.40 (m, 1H) | — | H21, H22b, H23 |
| | | 1.10 (m, 1H) | — | H21, H22a, H23 |
| 23 | 27.1 (t) | 1.40 (m, 2H) | — | H22a, H22b, H24a, |
| | | | | H24b |
| 24 | 38.1 (t) | 2.76 (m, 1H) | — | H23, H24b, H25 |
| | | 3.53 (m, 1H) | — | H23, H24a, H25 |
| 25 | — | 7.50 (dd, 1.2, 7.8 Hz, 1H) | — | H24a, H24b |
| 26 | — | 6.36 (d, 6.8 Hz, 1H) | 40 | H20 |
| tryptophan | | | | |
| 27 | 173.5 (s) | — | — | — |
| 28 | 53.8 (d) | 4.41 (ddd, 2.9, 9.6, 11.7 Hz, 1H) | — | H29a, H29b, H39 |
| 29 | 27.7 (t) | 2.90 (dd, 11.7, 13.7 1H) | 30, 31, 38 | H28, H29b |
| | | 3.30 (dd, 2.9, 13.7 Hz, 1H) | 30, 31, 38 | H28, H29a |
| 30 | 110.9 (s) | — | — | — |
| 31 | 124.9 (d) | 6.78 (bs, 1H) | 29, 30, 33, 38 | H32 |
| 32 | — | 11.00 (bs, 1H) | 30, 31, 33, 38 | H31 |
| 33 | 136.7 (s) | — | — | — |
| 34 | 111.3 (d) | 7.30 (d, 1.8 Hz, 1H) | 36, 38 | H36 |
| 35 | 125.8 (s) | — | — | — |
| 36 | 118.7 (d) | 6.93 (dd, 7.8, 1.8 Hz, 1H) | 38, 34 | H34, H37 |
| 37 | 118.3 (d) | 7.42 (d, 7.8 Hz, 1H) | 35, 33 | H36 |
| 38 | 125.5 (s) | — | — | — |
| 39 | | 8.64 (d, 9.6 Hz, 1H) | 1 | H28 |
| 40 | 157.5 (s) | — | — | — |
| arginine | | | | |
| 41 | — | 6.37 (d, 7.8 Hz, 1H) | 40 | H42 |
| 42 | 52.6 (d) | 4.05 (ddd, 5.9, 7.8, 7.8 Hz, 1H) | 43, 44, 48 | H41, H43a, H43b |
| 43 | 29.5 (t) | 1.50 (m, 1H) | — | H42, H43b, H44 |
| | | 1.67 (m, 1H) | — | H42, H43a, H44 |
| 44 | 25.1 (t) | 1.40 (m, 1H) | — | H43a, H43b, H45 |
| | | 1.19 (m, 1H) | | |
| 45 | 40.5 (t) | 3.06 (m, 2H) | 47 | H44, H46 |
| 46 | — | 7.50 (m, 1H) | — | H45 |
| 47 | 156.8 (s) | — | — | — |
| 48 | 174.3 (s) | — | — | — |

^{a}Chemical shifts determined from 2D heteronuclear experiments

TABLE 6
¹H (600 MHz), ¹³C (125 MHz), HMBC and COSY
NMR data for Compound 6 in d₆-DMSO

| | |
|---|---|
| | Compound 6 |
| 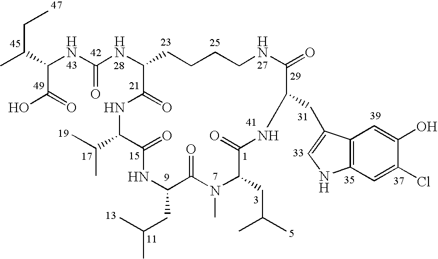 | |

| Atom No | ¹³C (mult)^{a} | | ¹H (mult, J Hz) | | ^{2,3}J_{CH} correlations | COSY |
|---|---|---|---|---|---|---|
| N-Methyl leucine | | | | | | |
| 1 | 169.4 | (s) | | — | — | — |
| 2 | 58.0 | (d) | 4.72 | (dd, 5.9, 8.8 Hz, 1H) | 1, 3, 4, 8, 7-NMe | H3a, H3b |
| 3 | 36.2 | (t) | 1.25 | (m, 1H) | 1, 2, 4 | H2, H3b, H4 |
| | | | 1.60 | (m, 1H) | 2, 4 | H2, H3a, H4 |
| 4 | 23.0 | (d) | 1.93 | (m, 1H) | 2, 3 | H3a, H3b, H5, H6 |
| 5 | 23.7 | (q) | 0.82 | (d, 6.8 Hz, 3H) | 3, 4, 6 | H4 |
| 6 | 24.0 | (q) | 0.82 | (d, 6.8 Hz, 3H) | 3, 4, 5 | H4 |
| NMe | 27.0 | (q) | 1.90 | (s, 3H) | 2, 8 | — |
| Leucine | | | | | | |
| 8 | 172.5 | (s) | | — | — | — |
| 9 | 47.8 | (d) | 4.70 | (ddd, 2.9, 4.9, 9.8 Hz, 1H) | — | H10a, H10b, H14 |
| 10 | 39.2 | (t) | 1.70 | (m, 1H) | — | H9, H10b, H11 |
| | | | 1.22 | (m, 1H) | — | H9, H10a, H11 |
| 11 | 27.0 | (d) | 1.82 | (m, 1H) | — | H10a, H10b, H12, |
| | | | | | | H13 |
| 12 | 21.0 | (q) | 0.84 | (d, 6.8 Hz, 3H) | 10, 11, 13 | H11 |
| 13 | 24.9 | (q) | 0.96 | (d, 6.8 Hz, 3H) | 10, 11, 12 | H11 |
| 14 | — | | 8.69 | (d, 4.9 Hz, 1H) | 9, 10, 15 | H9 |
| valine | | | | | | |
| 15 | 172.7 | (s) | | — | — | — |
| 16 | 57.8 | (d) | 3.92 | (dd, 5.8, 7.8 Hz, 1H) | — | H17, H20 |
| 17 | 29.7 | (d) | 1.95 | (m, 1H) | 16, 18, 19 | H16, H18, H19 |
| 18 | 19.4 | (q) | 0.85 | (d, 7.8 Hz, 3H) | 16, 17, 19 | H17 |
| 19 | 19.0 | (q) | 1.05 | (d, 7.8 Hz, 3H) | 16, 17, 18 | H17 |
| 20 | — | | 6.80 | (d, 5.9 Hz, 1H) | 16, 17, 19 | H16 |
| lysine | | | | | | |
| 21 | 172.5 | (s) | | — | — | — |
| 22 | 54.8 | (d) | 3.91 | (ddd, 5.9, 6.8, 6.8 Hz, 1H) | 19, 21, 22, 42 | H23, H28 |
| 23 | 31.5 | (t) | 1.60 | (m, 2H) | — | H22, H24a, H24b |
| 24 | 20.1 | (t) | 1.40 | (m, 1H) | — | H23, H24b, H25 |
| | | | 1.10 | (m, 1H) | — | H23, H24a, H25 |
| 25 | 28.1 | (t) | 1.40 | (m, 2H) | — | H24a, H24b, H26a, |
| | | | | | | H26b |
| 26 | 38.1 | (t) | 2.80 | (m, 1H) | 27 | H25, H26b, H27 |
| | | | 3.61 | (m, 1H) | — | H25, H26a, H27 |
| 27 | — | | 7.40 | (dd, 1.2, 7.8 Hz, 1H) | 27 | H26a, H26b |
| 28 | — | | 6.47 | (d, 5.9 Hz, 1H) | 42, 22, 23 | H22 |
| tryptophan | | | | | | |
| 29 | 171.6 | (s) | | — | — | — |
| 30 | 53.2 | (d) | 4.41 | (ddd, 2.9, 8.8, 11.7 Hz, 1H) | — | H31a, H31b, H41 |
| 31 | 27.9 | (t) | 2.90 | (dd, 11.7, 13.7 Hz, 1H) | 29, 33, 32, 30 | H30, H31b |
| | | | 3.40 | (dd, 2.9, 13.7 Hz, 1H) | 30, 32, 33 | H30, H31a |
| 32 | 109.5 | (s) | | — | — | — |
| 33 | 125.5 | (d) | 6.65 | (bs, 1H) | 29, 30, 35, 40 | H34 |
| 34 | — | | 10.64 | (bs, 1H) | 32, 33, 35, 40 | H33 |
| 35 | 130.4 | (s) | | — | — | — |
| 36 | 111.1 | (d) | 7.20 | (s, 1H) | 33, 37, 38, 40 | — |
| 37 | 115.0 | (s) | | — | — | — |
| 38 | 146.3 | (s) | | — | — | — |
| 39 | 102.3 | (d) | 7.00 | (s, 1H) | 35, 33, 32, 37, 38 | — |
| 40 | 126.0 | (s) | | — | — | — |
| 41 | — | | 8.77 | (d, 8.8 Hz, 1H) | 1 | H30 |
| 42 | 157.6 | (s) | | — | — | — |
| isoleucine | | | | | | |
| 43 | — | | 6.35 | (d, 7.8 Hz, 1H) | 42 | H44 |
| 44 | 56.9 | (d) | 4.06 | (dd, 5.9, 7.8 Hz, 1H) | 42, 45, 46, 48, 49 | H43, H45 |
| 45 | 36.8 | (d) | 1.70 | (m, 1H) | — | H44, H46b, H46a, |
| | | | | | | H48 |
| 46 | 24.7 | (t) | 1.40 | (m, 1H) | 44, 47, 48 | H46b, H47, H45 |
| | | | 1.15 | (m, 1H) | 44, 47, 48 | H47, H45, H46a |
| 47 | 11.7 | (q) | 0.82 | (t, 6.8 Hz, 3H) | 45, 46 | H46a, H46b |
| 48 | 15.4 | (q) | 0.84 | (d, 6.8 Hz, 3H) | 44, 45, 46 | H45 |
| 49 | 173.7 | (s) | | — | — | — |

^{a}Chemical shifts determined from 2D heteronuclear experiments

TABLE 7
¹H (600 MHz), ¹³C (125 MHz), HMBC and COSY
NMR data for Compound 7 in d₆-DMSO

| | |
|---|---|
| | Compound 7 |
| 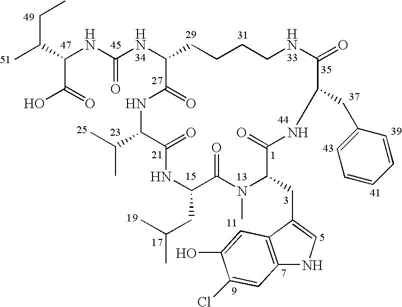 | |

| Atom No | ¹³C (mult)^{a} | | ¹H (mult, J Hz) | | ^{2,3}J_{CH} correlations | COSY |
|---|---|---|---|---|---|---|
| N-Methyl | | | | | | |
| tryptophan | | | | | | |
| 1 | 169.8 | (s) | | — | — | — |
| 2 | 61.0 | (d) | 4.66 | (dd, 2.6, 10.4 Hz, 1H) | 1, 3, 4, 14, 13-NMe | H3a, H3b |
| 3 | 22.3 | (t) | 2.73 | (m, 1H) | 1, 5, 4, 2, 12 | H2, H3b |
| | | | 3.07 | (m, 1H) | 2, 4, 5, 12 | H2, H3a |
| 4 | 108.9 | (s) | | — | — | — |
| 5 | 124.3 | (d) | 6.87 | (bs, 1H) | 3, 4, 7, 12 | H6 |
| 6 | — | | 10.66 | (bs, 1H) | 4, 5, 7, 12 | H5 |
| 7 | 130.7 | (s) | | — | — | — |
| 8 | 111.8 | (d) | 7.26 | (s, 1H) | 7, 9, 10, 12 | — |
| 9 | 115.8 | (s) | | — | — | — |
| 10 | 145.8 | (s) | | — | — | — |
| 11 | 102.7 | (d) | 6.98 | (s, 1H) | 4, 7, 9, 10, 12 | — |
| 12 | 126.8 | (s) | | — | — | — |
| NMe | 27.5 | (q) | 1.91 | (s, 3H) | 2, 14 | — |
| Leucine | | | | | | |
| 14 | 172.5 | (s) | | — | — | — |
| 15 | 46.9 | (d) | 4.21 | (ddd, 2.9, 4.9, 9.8 Hz, 1H) | 16, 21 | H16a, H16b, H20 |
| 16 | 36.9 | (t) | −0.50 | (dd, 11.7, 11.7 Hz, 1H) | 14, 17, 18 | H15, H16b, H17 |
| | | | 0.90 | (m, 1H) | — | H15, H16a, H17 |
| 17 | 24.8 | (d) | 1.40 | (m, 1H) | — | H16a, H16b, H18, |
| | | | | | | H19 |
| 18 | 19.7 | (q) | 0.26 | (d, 6.8 Hz, 3H) | 16, 17, 19 | H17 |
| 19 | 22.0 | (q) | 0.40 | (d, 6.8 Hz, 3H) | 16, 17, 18 | H17 |
| 20 | — | | 8.42 | (d, 4.3 Hz, 1H) | 15, 16, 21 | H15 |
| valine | | | | | | |
| 21 | 172.2 | (s) | | — | — | — |
| 22 | 57.6 | (d) | 3.79 | (dd, 6.9, 7.8 Hz, 1H) | 23, 24, 25 | H23, H26 |
| 23 | 30.0 | (d) | 1.90 | (m, 1H) | 22, 24, 25 | H22, H24, H25 |
| 24 | 18.9 | (q) | 0.86 | (d, 7.8 Hz, 3H) | 22, 23, 25 | H23 |
| 25 | 18.8 | (q) | 0.93 | (d, 7.8 Hz, 3H) | 22, 23, 24 | H23 |
| 26 | — | | 6.74 | (d, 6.9 Hz, 1H) | 22, 23, 27 | H22 |
| lysine | | | | | | |
| 27 | 171.9 | (s) | | — | — | — |
| 28 | 53.8 | (d) | 3.86 | (ddd, 5.9, 6.9, 6.8 Hz, 1H) | 27, 29, 30, 45 | H29, H34 |
| 29 | 31.3 | (t) | 1.54 | (m, 2H) | — | H28, H34 |
| 30 | 20.2 | (t) | 1.40 | (m, 1H) | — | H29, H30b, H31 |
| | | | 1.10 | (m, 1H) | — | H29, H30a, H31 |
| 31 | 18.2 | (t) | 1.40 | (m, 2H) | — | H30a, H30b, H32a, |
| | | | | | | H32b |
| 32 | 37.9 | (t) | 2.86 | (m, 1H) | 35 | H31, H32b, H33 |
| | | | 3.58 | (m, 1H) | 30, 31, 35 | H31, H32a, H33 |
| 33 | — | | 7.40 | (dd, 1.2, 7.8 Hz, 1H) | 32, 35 | H32a, H32b |
| 34 | — | | 6.43 | (d, 6.9 Hz, 1H) | 27, 29, 45 | H28 |
| phenylalanine | | | | | | |
| 35 | 171.0 | (s) | | — | — | — |
| 36 | 54.8 | (d) | 4.57 | (ddd, 2.9, 9.5, 11.7 Hz, 1H) | 1, 35, 37 | H37a, H37b, H44 |
| 37 | 37.9 | (t) | 2.75 | (dd, 11.7, 13.7 1H) | 35, 36, 38, 39, 43 | H36, H37b |
| | | | 3.40 | (dd, 2.9, 13.7 Hz, 1H) | 36, 38, 39, 43 | H36, H37a |
| 38 | 138.6 | (s) | | — | — | — |
| 39 | 128.9 | (d) | 7.07 | (d, 7.8 Hz, 1H) | 37, 38, 41, 43 | H40, H41 |
| 40 | 127.9 | (d) | 7.22 | (dd, 7.8, 7.8 Hz, 1H) | 38, 42 | H39, H41 |
| 41 | 126.2 | (d) | 7.15 | (t, 7.8 Hz, 1H) | 39, 43 | H40, H42 |
| 42 | 127.9 | (d) | 7.22 | (dd, 7.8, 7.8 Hz, 1H) | 38, 40 | H41, H43 |
| 43 | 128.29 | (d) | 7.07 | (d, 7.8 Hz, 1H) | 37, 38, 39, 41 | H42 |
| 44 | — | | 8.76 | (d, 9.5 Hz, 1H) | 1, 36, 37 | H36 |
| 45 | 157.3 | (s) | | — | — | — |
| isoleucine | | | | | | |
| 46 | — | | 6.28 | (d, 8.7 Hz, 1H) | 45, 47, 52 | H47 |
| 47 | 56.6 | (d) | 4.04 | (dd, 5.9, 7.8, 7.8 Hz, 1H) | 45, 48, 49, 51, 52 | H46, H48 |
| 48 | 36.9 | (d) | 1.71 | (m, 1H) | 47, 49, 50, 51 | H47, H49b, H51 |
| 49 | 24.5 | (t) | 1.35 | (m, 1H) | 47, 48, 50, 51 | H48, H49b, H50 |
| | | | 1.10 | (m, 1H) | 47, 48, 50, 51 | H48, H49a, H50 |
| 50 | 11.1 | (q) | 0.83 | (t, 6.8 Hz, 3H) | 48, 49 | H49a, H49b |
| 51 | 15.6 | (q) | 0.82 | (d, 6.8 Hz, 3H) | 47, 48, 49 | H48 |
| 52 | 173.8 | (s) | | — | — | — |

^{a}Chemical shifts determined from 2D heteronuclear experiments

TABLE 8
¹H (600 MHz), ¹³C (125 MHz) and COSY
NMR data for Compound 8 in d₆-DMSO

| | |
|---|---|
| | Compound 8 |
| 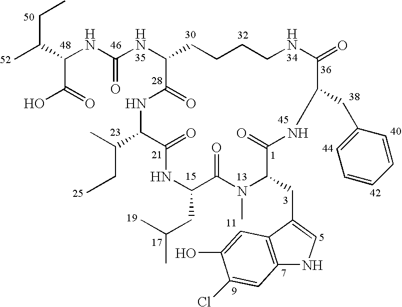 | |

| Atom No | ¹³C (mult)^{a} | | ¹H (mult, J Hz) | | COSY |
|---|---|---|---|---|---|
| N-Methyl | | | | | |
| tryptophan | | | | | |
| 1 | n.o. | | | — | — |
| 2 | 60.8 | (d) | 4.65 | (dd, 2.6, 9.9 Hz, 1H) | H3a, H3b |
| 3 | 21.9 | (t) | 2.73 | (m, 1H) | H2, H3b |
| | — | | 3.08 | (m, 1H) | H2, H3a |
| 4 | n.o. | | | — | — |
| 5 | 124.7 | (d) | 6.87 | (d, 1.9 Hz, 1H) | H6 |
| 6 | | | 10.66 | (bs, 1H) | H5 |
| 7 | n.o. | | | — | — |
| 8 | 111.5 | (d) | 7.23 | (s, 1H) | — |
| 9 | n.o. | | | — | — |
| 10 | n.o. | | | — | — |
| 11 | 103.4 | (d) | 6.94 | (s, 1H) | — |
| 12 | n.o. | | | — | — |
| NMe | 27.4 | (q) | 1.90 | (s, 3H) | — |
| Leucine | | | | | |
| 14 | n.o. | | | — | — |
| 15 | 47.4 | (d) | 4.18 | (ddd, 2.9, 4.9, 9.8 Hz, 1H) | H16a, H16b, H20 |
| 16 | 37.0 | (t) | −0.50 | (dd, 9.8, 9.8 Hz, 1H) | H15, H16b, H17 |
| | | | 0.91 | (m, 1H) | H15, H16a, H17 |
| 17 | 24.9 | (d) | 1.40 | (m, 1H) | H16a, H16b, H19, H18 |
| 18 | 19.5 | (q) | 0.22 | (d, 6.8 Hz, 3H) | H17 |
| 19 | 22.3 | (q) | 0.36 | (d, 6.8 Hz, 3H) | H17 |
| 20 | — | | 8.40 | (d, 4.8 Hz, 1H) | H15 |
| isoleucine | | | | | |
| 21 | n.o. | | | — | — |
| 22 | 55.8 | (d) | 3.93 | (dd, 7.8, 8.2 Hz, 1H) | H23, H27 |
| 23 | 37.0 | (d) | 1.72 | (m, 1H) | H22, H24a, H24b, H26 |
| 24 | 24.2 | (t) | 1.08 | (m, 1H) | H24b, H23, H25 |
| | | | 1.30 | (m, 1H) | H24a, H23, H25 |
| 25 | 12.0 | (q) | 0.82 | (d, 7.0 Hz, 3H) | H24a, H24b |
| 26 | 15.7 | (q) | 0.83 | (d, 7.0 Hz, 3H) | H23 |
| 27 | — | | 6.70 | (d, 6.9 Hz, 1H) | H22 |
| lysine | | | | | |
| 28 | n.o. | | | — | — |
| 29 | 54.3 | (d) | 3.85 | (ddd, 5.9, 6.8, 6.8 Hz, 1H) | H30, H35 |
| 30 | 31.8 | (t) | 1.54 | (m, 1H) | H29, H30b, H31a, H31b |
| | | | 1.72 | (m, 1H) | H29, H30a, H31a, H31b |
| 31 | 24.9 | (t) | 1.40 | (m, 1H) | H32, H31b, H30a, H30b |
| | | | 1.10 | (m, 1H) | H32, H31a, H30a, H30b |
| 32 | 28.1 | (t) | 1.40 | (m, 2H) | H31a, H31b, H33a, H33b |
| 33 | 38.0 | (t) | 2.80 | (m, 1H) | H32, H33b, H34 |
| | | | 3.55 | (m, 1H) | H32, H33a, H34 |
| 34 | — | | 7.43 | (dd, 1.2, 8.8 Hz, 1H) | H33a, H33b |
| 35 | — | | 6.45 | (d, 6.8 Hz, 1H) | H29 |
| phenylalanine | | | | | |
| 36 | n.o. | | | — | — |
| 37 | 54.5 | (d) | 4.58 | (ddd, 2.9, 8.8, 11.7 Hz, 1H) | H38a, H38b, H45 |
| 38 | 37.4 | (t) | 2.73 | (dd, 11.7, 11.7 Hz, 1H) | H37, H38b |
| | | | 3.37 | (dd, 2.9, 11.7 Hz, 1H) | H37, H38a |
| 39 | n.o. | | | — | — |
| 40 | 128.3 | (d) | 7.05 | (d, 7.8 Hz, 1H) | H41, H42 |
| 41 | 128.0 | (d) | 7.19 | (dd, 7.8, 7.8 Hz, 1H) | H40, H42 |
| 42 | 125.9 | (d) | 7.14 | (t, 7.8 Hz, 1H) | H41, H43 |
| 43 | 128.0 | (d) | 7.19 | (dd, 7.8, 7.8 Hz, 1H) | H42, H44 |
| 44 | 128.3 | (d) | 7.05 | (d, 7.8, Hz, 1H) | H43, H42 |
| 45 | — | | 8.68 | (d, 8.8 Hz, 1H) | H37 |
| 46 | n.o. | | | — | — |
| isoleucine | | | | | |
| 47 | — | | 6.29 | (d, 8.8 Hz, 1H) | H48 |
| 48 | 56.3 | (d) | 4.01 | (dd, 4.9, 7.8, Hz, 1H) | H47, H49 |
| 49 | 38.3 | (d) | 1.71 | (m, 1H) | H48, H50, H50b, H52 |
| 50 | 22.8 | (t) | 1.38 | (m, H) | H50b, H49, H51 |
| | | | 1.01 | (m, 1H) | H50a, H49, H51 |
| 51 | 11.4 | (q) | 0.79 | (t, 6.8 Hz, 3H) | H50a, H50b |
| 52 | 15.8 | (q) | 0.79 | (d, 6.8 Hz, 3H) | H49 |
| 53 | n.o. | | | — | — |

^{a}Chemical shifts determined from 2D heteronuclear experiments
n.o. = not observed

TABLE 9
¹H (600 MHz), ¹³C (125 MHz), HMBC and COSY
NMR data for Compound 9 in d₆-DMSO

| | |
|---|---|
| | Compound 9 |
| 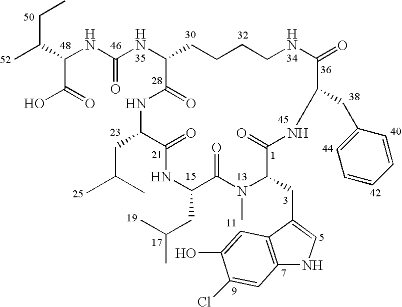 | |

| Atom No | ¹³C (mult)^{a} | | ¹H (mult, J Hz) | | ^{2,3}H_{CH} correlations | COSY |
|---|---|---|---|---|---|---|
| N-Methyl | | | | | | |
| tryptophan | | | | | | |
| 1 | 169.5 | (s) | | — | — | — |
| 2 | 60.8 | (d) | 4.69 | (dd, 2.6, 10.4 Hz, 1H) | 1 | H3a, H3b |
| 3 | 21.7 | (t) | 2.76 | (m, 1H) | 2, 4, 12 | H2, H3b |
| | | | 3.04 | (m, 1H) | 2, 4, 12 | H2, H3a |
| 4 | 108.9 | (s) | | — | — | — |
| 5 | 124.3 | (d) | 6.88 | (bs, 1H) | 4, 7, 12 | H6 |
| 6 | | | 10.66 | (bs, 1H) | 4, 5, 7, 12 | H5 |
| 7 | 130.2 | (s) | | — | — | — |
| 8 | 111.8 | (d) | 7.27 | (s, 1H) | 9, 10, 12 | — |
| 9 | 115.8 | (s) | | — | — | — |
| 10 | 145.9 | (s) | | — | — | — |
| 11 | 102.7 | (d) | 6.99 | (s, 1H) | 4, 7, 9, 10 | — |
| 12 | 126.1 | (s) | | — | — | — |
| NMe | 27.4 | (q) | 1.91 | (s, 3H) | 2, 14 | — |
| Leucine | | | | | | |
| 14 | 172.5 | (s) | | — | — | — |
| 15 | 46.7 | (d) | 4.22 | (ddd, 2.9, 4.9, 9.8 Hz, 1H) | — | H16a, H16b, H20 |
| 16 | 37.4 | (t) | −0.49 | (dd, 9.8, 9.8 Hz, 1H) | 18 | H15, H16b, H17 |
| | | | 0.95 | (m, 1H) | — | H15, H16a, H17 |
| 17 | 23.1 | (d) | 1.40 | (m, 1H) | — | H16a, H16b, H19, |
| | | | | | | H18 |
| 18 | 19.7 | (q) | 0.25 | (d, 6.8 Hz, 3H) | 16, 17, 19 | H17 |
| 19 | 22.3 | (q) | 0.42 | (d, 6.8 Hz, 3H) | 16, 17, 18 | H17 |
| 20 | — | | 8.47 | (d, 4.3 Hz, 1H) | 21 | H15 |
| leucine | | | | | | |
| 21 | 173.5 | (s) | | — | — | — |
| 22 | 50.7 | (d) | 4.03 | (td, 7.8, 6.9 Hz, 1H) | 21, 23 | H23, H27 |
| 23 | 39.7 | (t) | 1.46 | (m, 2H) | — | H22, H24 |
| 24 | 23.3 | (d) | 1.67 | (m, 1H) | 15, 16 | H23, H25, H26 |
| 25 | 21.6 | (q) | 0.82 | (d, 7.0 Hz, 3H) | 23, 24, 26 | H24 |
| 26 | 22.8 | (q) | 0.88 | (d, 7.0 Hz, 3H) | 23, 24, 25 | H24 |
| 27 | — | | 6.86 | (d, 6.9 Hz, 1H) | 28 | H22 |
| lysine | | | | | | |
| 28 | 172.2 | (s) | | — | — | H30, H35 |
| 29 | 54.4 | (d) | 3.88 | (ddd, 5.9, 6.8, 6.8 Hz, 1H) | 28, 30, 31 | H29, H31a, H31b |
| 30 | 32.1 | (t) | 1.54 | (m, 2H) | — | H30, H31b, H32 |
| 31 | 20.2 | (t) | 1.40 | (m, 1H) | — | H30, H31a, H32 |
| | | | 1.10 | (m, 1H) | — | H30, H22a, H23 |
| 32 | 28.1 | (t) | 1.42 | (m, 2H) | — | H31a, H31b, H33a, |
| | | | | | | H33b |
| 33 | 38.3 | (t) | 2.84 | (m, 1H) | — | H32, H33b, H34 |
| | | | 3.57 | (m, 1H) | — | H32, H33a, H34 |
| 34 | — | | 7.38 | (dd, 1.2, 7.8 Hz, 1H) | — | H33a, H33b |
| 35 | — | | 6.35 | (d, 6.8 Hz, 1H) | 46 | H29 |
| phenylalanine | | | | | | |
| 36 | 171.4 | (s) | | — | — | — |
| 37 | 54.5 | (d) | 4.52 | (ddd, 2.9, 8.8, 11.7 Hz, 1H) | 36 | H38a, H38b, H45 |
| 38 | 37.9 | (t) | 2.74 | (dd, 11.7, 13.7 Hz, 1H) | 39, 40, 44 | H37, H38b |
| | | | 3.55 | (dd, 2.9, 13.7 Hz, 1H) | 28, 27, 30, 31, 38 | H27, H38a |
| 39 | 138.3 | (s) | | — | — | — |
| 40 | 128.7 | (d) | 7.08 | (d, 8.0 Hz, 1H) | 42, 44 | H41, H42 |
| 41 | 129.2 | (d) | 7.23 | (dd, 8.0, 8.0 Hz, 1H) | 39, 43 | H40, H42 |
| 42 | 126.6 | (d) | 7.17 | (t, 8.0 Hz, 1H) | 40, 44 | H41, H43, H40, |
| | | | | | | H44 |
| 43 | 129.2 | (d) | 7.23 | (dd, 8.0, 8.0 Hz, 1H) | 39, 41 | H42, H44 |
| 44 | 128.7 | (d) | 7.08 | (d, 8.0 Hz, 1H) | 40, 38, 42 | H43, H42 |
| 45 | — | | 8.71 | (d, 8.8 Hz, 1H) | 1 | H37 |
| 46 | 157.0 | (s) | | — | — | — |
| isoleucine | | | | | | |
| 47 | — | | 6.26 | (d, 8.7 Hz, 1H) | — | H48 |
| 48 | 56.9 | (d) | 4.03 | (dd, 5.9, 7.8, 7.8 Hz, 1H) | 46, 49, 50, 52, 53 | H47, H49 |
| 49 | 37.6 | (d) | 1.70 | (m, 1H) | — | H48, H50b, H50a, |
| | | | | | | H52 |
| 50 | 24.6 | (t) | 1.35 | (m, 1H) | 48, 49, 51, 52 | H49, H50a, H50b, |
| | | | | | | H51 |
| | | | 1.10 | (m, 1H) | 49, 51, 52 | H49, H50a, H50b, |
| | | | | | | H51 |
| 51 | 11.7 | (q) | 0.86 | (t, 6.8 Hz, 3H) | 49, 50 | H50a, H50b |
| 52 | 15.8 | (q) | 0.85 | (d, 6.8 Hz, 3H) | 48, 49, 50 | H49 |
| 53 | 173.8 | (s) | | — | — | — |

^{a}Chemical shifts determined from 2D heteronuclear experiments

TABLE 10
¹H (600 MHz), ¹³C (125 MHz), HMBC and COSY
NMR data for Compound 10 in d₆-DMSO

| | |
|---|---|
| | Compound 10 |
| 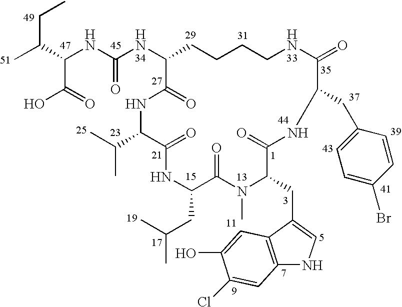 | |

| Atom No | ¹³C (mult)^{a} | | ¹H (mult, J Hz) | | ^{2,3}J_{CH} correlations | COSY |
|---|---|---|---|---|---|---|
| N-Methyl | | | | | | |
| tryptophan | | | | | | |
| 1 | 169.8 | (s) | | — | — | — |
| 2 | 60.9 | (d) | 4.66 | (dd, 2.9, 10.7 Hz, 1H) | 1, 3, 4, 14, 13-NMe | H3a, H3b |
| 3 | 21.9 | (t) | 2.77 | (m, 1H) | 2, 4, 5 | H2 ,H3b |
| | | | 3.07 | (m, 1H) | 2, 4, 5 | H2, H3a |
| 4 | 109.3 | (s) | | — | — | — |
| 5 | 126.1 | (d) | 6.89 | (d, 2.0 Hz, 1H) | 4, 7, 12 | H6 |
| 6 | — | | 10.68 | (bs, 1H) | 4, 5, 7, 12 | H5 |
| 7 | 130.5 | (s) | | — | — | — |
| 8 | 111.8 | (d) | 7.26 | (s, 1H) | 7, 9, 10, 12 | — |
| 9 | 115.8 | (s) | | — | — | — |
| 10 | 146.2 | (s) | | — | — | — |
| 11 | 103.4 | (d) | 6.98 | (s, 1H) | 4, 7, 9 | — |
| 12 | 126.8 | (s) | | — | — | — |
| NMe | 27.3 | (q) | 1.97 | (s, 3H) | 2, 14 | — |
| Leucine | | | | | | |
| 14 | 171.9 | (s) | | — | — | — |
| 15 | 46.8 | (d) | 4.21 | (ddd, 2.9, 4.9, 11.7 Hz, 1H) | — | H16a, H16b, H20 |
| 16 | 37.2 | (t) | −0.48 | (dd, 11.7, 11.7 Hz, 1H) | — | H15, H16b, H17 |
| | | | 0.95 | (m, 1H) | — | H15, H16a, H17 |
| 17 | 23.3 | (d) | 1.40 | (m, 1H) | — | H16a, H16b, H19, |
| | | | | | | H18 |
| 18 | 19.5 | (q) | 0.27 | (d, 6.8 Hz, 3H) | 16, 17, 19 | H17 |
| 19 | 21.3 | (q) | 0.41 | (d, 6.8 Hz, 3H) | 16, 17, 18 | H17 |
| 20 | — | | 8.42 | (d, 4.9 Hz, 1H) | 15, 16, 21 | H15 |
| leucine | | | | | | |
| 21 | 172.9 | (s) | | — | — | — |
| 22 | 57.7 | (d) | 3.77 | (dd, 6.8, 7.8 Hz, 1H) | 21, 23, 24, 25 | H23, H26 |
| 23 | 29.8 | (t) | 1.88 | (m, 2H) | — | H22, H23, H24 |
| 24 | 18.9 | (q) | 0.84 | (d, 7.0 Hz, 3H) | 22, 23, 25 | H23 |
| 25 | 18.9 | (q) | 0.93 | (d, 7.0 Hz, 3H) | 22, 23, 24 | H23 |
| 26 | — | | 6.74 | (d, 6.9 Hz, 1H) | 23, 28 | H22 |
| lysine | | | | | | |
| 27 | 172.2 | (s) | | — | — | — |
| 28 | 54.5 | (d) | 3.84 | (ddd, 5.9, 6.8, 6.8 Hz, 1H) | 20, 28, 29, 45 | H29, H34 |
| 29 | 31.5 | (t) | 1.54 | (m, 2H) | — | H28, H30a, H30b |
| 30 | 20.2 | (t) | 1.40 | (m, 1H) | — | H29, H30b, H31 |
| | | | 1.10 | (m, 1H) | — | H29, H30a, H31 |
| 31 | 28.2 | (t) | 1.42 | (m, 2H) | — | H30a, H30b, H32a, |
| | | | | | | H32b |
| 32 | 38.3 | (t) | 2.85 | (m, 1H) | — | H31, H32b, H33 |
| | | | 3.57 | (m, 1H) | 30, 31 | H31, H32a, H33 |
| 33 | — | | 7.46 | (dd, 1.2, 7.0 Hz, 1H) | 35 | H32a, H32b |
| 34 | — | | 6.41 | (d, 6.8 Hz, 1H) | 29, 28, 45 | H28 |
| phenylalanine | | | | | | |
| 35 | 170.4 | (s) | | — | — | — |
| 36 | 54.1 | (d) | 4.52 | (ddd, 2.9, 8.8, 11.7 Hz, 1H) | 35 | H37a, H37b, H44 |
| 37 | 37.2 | (t) | 2.72 | (dd, 11.7, 13.7 1H) | 36, 38, 39, 43 | H36, H37b |
| | | | 3.36 | (dd, 2.9, 13.7 Hz, 1H) | 36, 38, 39, 43 | H36, H37a |
| 38 | 137.9 | (s) | | — | — | — |
| 39 | 131.4 | (d) | 7.01 | (d, 7.8 Hz, 1H) | 37, 41, 43 | H40 |
| 40 | 130.4 | (d) | 7.37 | (d, 7.8 Hz, 1H) | 42, 38 | H39 |
| 41 | 119.2 | (s) | | — | — | — |
| 42 | 130.4 | (d) | 7.39 | (d, 7.8 Hz, 1H) | 40, 38 | H43 |
| 43 | 131.4 | (d) | 7.08 | (d, 7.8 Hz, 1H) | 37, 39, 41 | H42 |
| 44 | — | | 8.81 | (d, 8.8 Hz, 1H) | — | H36 |
| 45 | 157.3 | (s) | | — | — | — |
| isoleucine | | | | | | |
| 46 | — | | 6.26 | (d, 8.8 Hz, 1H) | 45, 47 | H47 |
| 47 | 57.2 | (d) | 4.04 | (dd, 4.9, 8.8, 7.8 Hz, 1H) | 45, 48, 49, 51, 52 | H48, H46 |
| 48 | 37.2 | (d) | 1.70 | (m, 1H) | — | H47, H49b, H49a |
| 49 | 25.1 | (t) | 1.33 | (m, 1H) | 47, 48, 50, 51 | H49a, H48, H50 |
| | | | 1.07 | (m, 1H) | 47, 48, 50, 51 | H49b, H48, H50 |
| 50 | 11.4 | (q) | 0.83 | (t, 6.8 Hz, 3H) | 48, 49 | H49a, H49b |
| 51 | 15.8 | (q) | 0.83 | (d, 6.8 Hz, 3H) | 47, 48, 49 | H48 |
| 52 | 174.5 | (s) | | — | — | — |

^{a}Chemical shifts determined from 2D heteronuclear experiments

### EXAMPLE 2

This Example describes the isolation of Compound 11.

### General Experimental Procedures

Water was Milli-Q filtered, while all other solvents used were Omnisolv. A Hypersil BDS basic C18 5 uM, 21.2 mm×150 mm, column were used for preparative HPLC. NMR spectra were recorded on a Varian Inova 600 or 500 MHz NMR spectrometer. Samples were dissolved in d₆-DMSO and chemical shifts were calculated relative to the solvent peak (DMSO ¹H □ 2.50 and ¹³C 39.5 ppm). Mass spectra were measured on a Fisons VG Platform II, using positive electrospray ionisation mode. The elution solvent was a mixture acetonitrile/water 50% at 0.1 ml/min.

### Animal Material

Six sponge samples of Candidaspongia flabellata were collected by SCUBA diving at Outer Gneering, Sunshine Coast, Old Reef, Fairfax Is and Chauvel Reef, Queensland, Australia and voucher samples (G315106, G314580, G314025, G315402, G318260, G317513) were lodged at the Queensland Museum, Brisbane, Australia.

### Extraction and Isolation

The freeze-dried sponge materials (529 g) were ground and exhaustively extracted with methanol to afford six methanol extracts. The methanol crude extracts underwent a series of partitions: MeOH/n-hexane, H₂O:MeOH (4:1)/DCM, H₂O:MeOH (4:1)/EtOAc. Bioactivity was spread in the H₂O:MeOH (4:1) and EtOAc layers. The H₂O:MeOH (4:1) and EtOAc layers were combined for all six biota and then partitioned with H₂O/butanol. The activity was in the butanol layer (900 mg), which then underwent countercurrent chromatography {H₂O/MeOH/EtOAc (4:1:5)}, upper layer mobile phase. The very early eluting fractions, 13-24, were combined (325 mg) and partitioned n-hexane:EtOAc:MeOH:H₂O (1:1:1:1). The bioactive aqueous layer (150 mg) was then chromatographed further by counter current chromatography {(CHCl₃:MeOH:H₂O (7:13:8)}, lower layer mobile phase. The early eluting active fractions, 25-32, were combined to give 85 mg of material. This underwent a final purification step by HPLC (Hypersil BDS C18) using a 30 min H₂O/MeCN gradient from H₂O (containing 1% TFA) to MeCN (containing 1% TFA). This yielded 0.4 mg of Compound 11 eluting after 18.2 mins.
Compound 11: MS: (positive ESI)) [M+H]⁺ m/z 1003.0 (100), 1004.4 (72), 1005.4 (75), 1006.3 (32). ¹H and ¹³C NMR (d₆-DMSO): see Table 11.

Compound 11 was also identified as a cyclic peptide after detailed studies, including ¹H, ¹³C, gHSQC, gHMBC, and gCOSY experiments.

TABLE 11
¹H (600 MHz), ¹³C (125 MHz), HMBC and COSY
NMR data for Compound 11 in d₆-DMSO

| | |
|---|---|
| | Compound 11 |
| 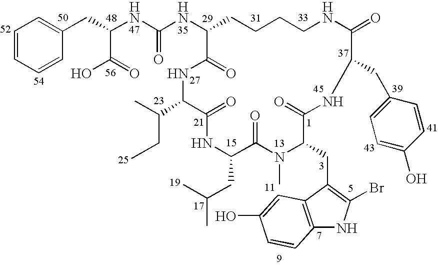 | |

| Atom No | ¹³C (mult)^{a} | | ¹H (mult, J Hz) | | ^{2,3}J_{CH} correlations | COSY |
|---|---|---|---|---|---|---|
| H-Methyl | | | | | | |
| tryptophan | | | | | | |
| 1 | n.o. | | | — | — | — |
| 2 | 60.0 | (d) | 4.70 | (bd, 10.8 Hz, 1H) | — | H3a, H3b |
| 3 | 22.4 | (t) | 2.71 | (dd, 14.5, 10.8 Hz, 1H) | — | H2, H3b |
| | | | 3.14 | (d, 14.5 Hz, 1H) | — | H3a |
| 4 | n.o. | | | — | — | — |
| 5 | 108.9 | (s) | | — | — | — |
| 6 | — | | 11.33 | (s, 1H) | 4, 7, 12 | — |
| 7 | 130.8 | (s) | | — | — | — |
| 8 | 111.0 | (d) | 7.05 | (bd, 8.0 Hz, 1H) | 12, 10 | H9 |
| 9 | 111.8 | (d) | 6.60 | (bd, 8.0 Hz, 1H) | — | H8 |
| 10 | 150.8 | (s) | | — | — | — |
| 11 | 101.8 | (d) | 6.82 | (bs, 1H) | 7, 10 | — |
| 12 | 128.1 | (s) | | — | — | — |
| NMe | 28.5 | (q) | 2.10 | (s, 3H) | 2 | — |
| Leucine | | | | | | |
| 14 | 172.4 | (s) | | — | — | — |
| 15 | 46.8 | (d) | 4.16 | (m, 1H) | — | H16a, H16b, H20 |
| 16 | 36.6 | (t) | 0.32 | (bt, 11.0 Hz, 1H) | 15 | H16b, H17 |
| | | | 0.96 | (m, 1H) | — | H15, H16a |
| 17 | 22.4 | (d) | ^{a}1.42 | (m, 1H) | — | — |
| 18 | 19.0 | (q) | 0.22 | (d, 6.6 Hz, 3H) | 16, 17, 19 | H17 |
| 19 | 22.1 | (q) | 0.41 | (d, 6.6 Hz, 3H) | 16, 17, 18 | H17 |
| 20 | — | | 8.38 | (d, 4.8 Hz, 1H) | 14 | H15 |
| Isoleucine | | | | | | |
| 21 | 171.6 | (s) | | — | — | — |
| 22 | 55.7 | (d) | 3.99 | (t, 6.8 Hz, 1H) | 23, 26 | H23, H27 |
| 23 | 35.7 | (d) | 1.76 | (m, 1H) | 21 | H22, H24a, H26 |
| 24 | 24.7 | (t) | 1.10 | (m, 1H) | — | H23, H24b, H25 |
| | | | ^{a}1.44 | (m, 1H) | — | H24a, H25 |
| 25 | 11.2 | (q) | 0.85 | (t, 7.2 Hz, 3H) | 23, 24 | H24a, H24b |
| 26 | 14.2 | (q) | 0.81 | (d, 6.6 Hz, 3H) | 22 | H23 |
| 27 | — | | 6.78 | (d, 6.8 Hz, 1H) | — | H22 |
| Lysine | | | | | | |
| 28 | 172.4 | (s) | | — | — | — |
| 29 | 54.3 | (d) | 3.85 | (ddd, 7.0, 6.5, 5.0 Hz, 1H) | 28 | H30a, H30b, H35 |
| 30 | 31.0 | (t) | 1.52 | (m, 1H) | — | H29, H31a |
| | | | 1.60 | (m, 1H) | — | H29, H31b |
| 31 | 20.1 | (t) | 1.14 | (m, 1H) | — | H30a |
| | | | 1.25 | (m, 1H) | — | H30b |
| 32 | 26.6 | (t) | 1.38 | (m, 1H) | — | H33b |
| | | | 1.41 | (m, 1H) | — | — |
| 33 | 37.8 | (t) | 2.85 | (m, 1H) | — | H34 |
| | | | 3.52 | (m, 1H) | — | H34, H32a |
| 34 | — | | 7.35 | (m, 1H) | — | H33a, H33b |
| 35 | — | | 6.48 | (d, 7.0 Hz, 1H) | — | H29 |
| Tyrosine | | | | | | |
| 36 | n.o. | | | — | — | — |
| 37 | 54.7 | (d) | 4.50 | (ddd, 11.7, 9.0, 4.9 Hz, 1H) | — | H38a, H38b, H45 |
| 38 | 36.5 | (t) | 2.62 | (bt, 13.0 Hz, 1H) | 39 | H37, H38b |
| | | | ^{a}3.23 | (m, 1H) | 39 | H37, H38a |
| 39 | 130.0 | (s) | | — | — | — |
| 40 | 128.5 | (d) | 6.87 | (d, 7.5 Hz, 1H) | 38, 39, 42 | H41 |
| 41 | 114.8 | (d) | 6.62 | (d, 7.5 Hz, 1H) | 40, 42, 44 | H40 |
| 42 | 156.0 | (s) | | — | — | — |
| 43 | 114.8 | (d) | 6.62 | (d, 7.5 Hz, 1H) | 40, 42, 44 | H44 |
| 44 | 128.5 | (d) | 6.87 | (d, 7.5 Hz, 1H) | 38, 39, 42 | H43 |
| 45 | — | | 8.54 | (d, 9.0 Hz, 1H) | — | H37 |
| 46 | n.o. | | | — | — | |
| Phenylalanine | | | | | | |
| 47 | — | | 6.26 | (d, 8.0 Hz, 1H) | — | H48 |
| 48 | 53.4 | (d) | 4.36 | (ddd, 8.0, 7.5, 5.2 Hz, 1H) | 56, 49 | H49a, H49b, H47 |
| 49 | 37.2 | (t) | 2.86 | (dd, 13.8, 7.5 Hz, 1H) | 56, 55, 51, 50, 48 | H48 |
| | | | 2.99 | (dd, 13.8, 5.2 Hz, 1H) | 56, 55, 51, 50, 48 | H48 |
| 50 | 137.5 | (s) | | — | — | — |
| 51 | 129.0 | (d) | 7.16 | (d, 7.5 Hz, 1H) | 53, 49 | H52, H54 |
| 52 | 128.0 | (d) | 7.27 | (t, 7.5 Hz, 1H) | 50 | H51, H55 |
| 53 | 126.2 | (d) | 7.20 | (t, 7.5 Hz, 1H) | 51, 55 | — |
| 54 | 128.0 | (d) | 7.27 | (t, 7.5 Hz, 1H) | 50 | H51, H55 |
| 55 | 129.0 | (d) | 7.16 | (d, 7.5 Hz, 1H) | 53, 49 | H52, H54 |
| 56 | 173.8 | (s) | | — | — | — |
| OH | — | | 8.71 | (s, 1H) | — | — |
| OH | — | | 9.13 | (s, 1H) | — | — |

^{a}Chemical shift estimated from 2D NMR experiments
n.o. = not observed.

### EXAMPLE 3

This Example describes the synthesis of Compound 12.

### General Experimental Procedures

High resolution mass spectra were recorded on a Micromass LCT mass spectrometer equipped with an electrospray interface (LC-HRMS). ¹H NMR measurements were performed on Varian UNITY plus 400, 500 and 600 spectrometers, operating at ¹H frequencies of 400, 500 and 600 MHz respectively. NMR spectra were recorded in d6-DMSO with chemical shifts given in ppm with the solvent as internal standard.

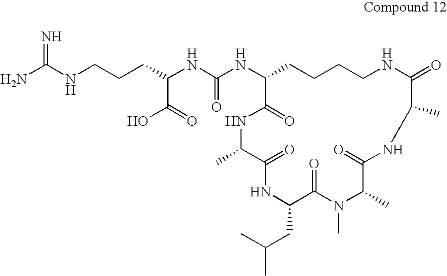

### Synthesis of Compound 12

Compound 12 was prepared according to a literature procedure (Marsh and Bradley, J. Org. Chem., 1997, 62, 6199-6203) with the following modifications: Fmoc-L-Arg-N^{ω,ω′}-(Boc)₂-OH was first coupled to the resin/linker. After removal of the Fmoc group, the free amine was coupled with N^{α}-(4-nitrophenyloxycarbonyl)-N^{ε}-(9-fluorenylmethoxycarbonyl)-D-lysine allyl ester. Fmoc peptide synthesis continued on the side chain of the lysine residue using Fmoc-L-Ala followed by Fmoc-L-N-MeAla, Fmoc-L-Leu and Fmoc-L-Ala. Allyl ester and Fmoc removal was followed by cyclization and finally cleavage from the resin/linker. Purification of the residue by reversed-phase HPLC (Ace C8 column, linear gradient 5%→95% MeCN in 0.1 M aqueous NH₄OAc) gave Compound 12 (1.8 mg, 1.3%).

¹H NMR (500 MHz, d₆-DMSO): □ 9.2 (broad s, 1H), 8.66 (d, 1H), 8.52 (d, 1H), 7.4-8.0 (broad signal, 4H), 7.47 (dd, 1H), 7.10 (d, 1H), 6.56 (d, 1H), 6.08 (d, 1H), 4.77-4.83 (m, 1H), 4.70-4.77 (m, 1H), 4.23 (qd, 1H), 4.07 (qd, 1H), 3.88-3.98 (m, 1H), 3.65-3.75 (m, 1H), 3.47-3.52 (m, 1H), 3.03 (broad t, 2H), 2.71-2.78 (m, 1H), 2.52 (s, 3H), 1.78-1.84 (m, 1H), 1.68-1.79 (m, 1H), 1.30-1.65 (m, 12H), 1.15-1.23 (m, 2H), 1.18 (two d, 6H), 0.94 (d, 3H), 0.93 (d, 3H), 0.89 (d, 3H), 0.88 (d, 3H).

HRMS (ESI) calculated for C₃₂H₅₉N₁₀O₈ 711.4517 (M+H)⁺, found 711.4525.

### EXAMPLE 4

This Example describes the synthesis of Compounds 1 and 13 to 16.

### Synthesis of Compound 1

a) Synthesis of Intermediate A

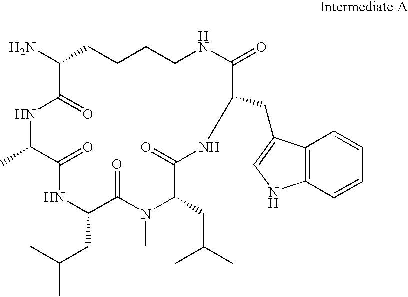

TFA (2 mL) was added to Boc-D-Lys(Fmoc)-OAllyl (2.86 g, 5.6 mmol) and left to stand for 5 min. The TFA was then removed by a stream of dry nitrogen to afford H-D-Lys(Fmoc)-OAllyl which was dried on a high vacuum line for 2 h to remove all traces of TFA. 2-Chlorotrityl resin (1 g, 1.4 mmol) was pre-swelled in DCM (10 mL) for 1 h. The resin was drained and a solution of H-D-Lys(Fmoc)-OAllyl (2.30 g, 5.64 mmol) and DIEA (729 mg, 982 μL, 5.64 mmol) in DCM (10 mL) was added and the reaction mixture shaken for 1 h. Further DIEA (1.46 g, 1.95 mL, 11.3 mmol) was added to the resin and the reaction mixture shaken for a further 1 h. Methanol (1 mL) was added to end-cap any unreacted resin and the reaction mixture shaken for a further 1 h. The resin was filtered and washed with DMF (2×5 mL), DCM (2×5 mL) and DMF (2×5 mL). The resin was subjected to Fmoc-solid phase peptide synthesis (SPPS) using the following conditions:
(i) Fmoc deprotection: 20% piperidine in DMF (2×10 mL) for 2 min followed by washing with DMF (4×5 mL), DCM (4×5 mL) and DMF (4×5 mL).
(ii) Coupling conditions: In all couplings the solution of the coupling reagent in DMF is added to the Fmoc-amino acid. This solution is added to the resin followed by DIEA. (a) Fmoc-Trp(Boc)-OH (2.95 g, 5.6 mmol), HBTU (0.5 M solution, 11.2 mL) and DIEA (0.975 mL, 5.6 mmol) 20 min. (b) Fmoc-N-Me-Leu-OH (2.06 g, 5.6 mmol), HBTU (0.5 M solution, 11.2 MnL) and DIEA (0.975 mL, 5.6 mmol) 20 min. (c) Fmoc-Leu-OH (1.98 g, 5.6 mmol), HOBt (756 mg, 5.6 mmol), HATU (2.13 g, 5.6 mmol) and DIEA (314 μL, 1.8 mmol) in DMF (10 mL) 3 h. (d) Fmoc-Ala-OH (1.74 g, 5.6 mmol), HBTU (0.5 M solution, 11.2 mL) and DIEA (0.975 mL, 5.6 mmol) 20 min. Following all couplings the resin was filtered and washed with DMF (4×5 mL), DCM (4×5 mL) and DMF (4×5mL). All couplings except for (c) were monitored using the ninhydrin test, coupling (c) was monitored using a bromophenol blue test. All couplings were also monitored by MS by cleaving a small amount of resin (5 mg) with 100% TFA for 5 min, the filtrate from the resin was then analysed by MS.

A solution of Pd(PPh₃)₄ (1.62 g, 1.4 mmol) and dimedone (1.96 g, 14 mmol) in THF:DCM (1:1, 50 mL) was sparged with nitrogen gas for 10 min., added to the resin and the mixture shaken for 16 h. The reaction mixture was filtered and washed with DCM (3×5 mL), DMF (3×5 mL) a solution of 0.5% DIEA and 0.5% diethyldithiocarbamic acid sodium salt in DMF (3×5 mL) and DMF (3×5mL). The resin was treated with 20% piperidine in DMF (2×10 mL) for 2 min. followed by washing with DMF (4×5 mL), DCM (4×5 mL), 10% pyridinium hydrochloride in DCM:DMF (1:1, 4×5 mL) and DMF (4×5 mL). A solution of PyBroP (718 mg, 1.54 mmol) and DIEA (1 mL, 5.74 mmol) in DCM:DMF (1:1, 10 mL) was added to the resin and the mixture shaken for 3 h after which a ninhydrin test was negative. The cyclic peptide was cleaved from the resin by treatment with 50% TFA in DCM (20 mL) for 1 h. The resin was filtered, washed with TFA (2×5 mL) and DCM (2×5 mL), concentrated to dryness, re-dissolved in MeCN:H₂O (0.1% TFA) and lyophilised to afford crude Intermediate A (435 mg, 50% based on the 2-chlorotrityl resin). Purification by RPHPLC (95:5 H₂O (1% TFA):MeCN (1% TFA) to 2:3 H₂O 1% TFA):MeCN (1% TFA)) over 60 min afforded Intermediate A (0.417 g, 3.6%).

b) Allyl-N²-[(9H-fluoren-9-ylmethoxy)carbonyl]-N⁵-{imino[(2, 2,4,6,7-pentamethyl-2,3-dihydro-1-benzofuran-5-yl)amino]methyl}ornithinate

N²-[(9H-fluoren-9-ylmethoxy)carbonyl]-N⁵-{imino[(2,2,4,6,7-pentamethyl-2,3-dihydro-1-benzofuran-5-yl)amino]methyl}ornithine (1.0 g, 1.54 mmol) was dissolved in DMF (5 mL). Caesium carbonate (377 mg, 1.16 mmol) was added and the reaction mixture stirred for 1 h. Allyl bromide (0.913 mL, 10.8 mmol) was then added and stirring was continued for a further 1 h resulting in a milky white solution. Water (25 mL) was added and the reaction mixture acidified with 2M KHSO₄. DCM (50 mL) was added and the phases separated. The aqueous phase was washed with DCM (2×50 mL) and the combined organics washed with brine (50 mL), dried (MgSO₄), filtered and concentrated to dryness to afford allyl-N²-[(9H-fluoren-9-ylmethoxy)carbonyl]-N⁵-{imino[(2,2,4,6,7-pentamethyl-2,3-dihydro-1-benzofuran-5-yl)amino]methyl}ornithinate as colourless foam (857 mg, 81%).

¹HNMR (CDCl₃, 500 MHz): □ 1.43 (s, 6H), 1.59 (m, 2H), 1.73 (m, 1H), 1.86 (m, 1H), 2.09 (s, 3H), 2.52 (s, 3H), 2.61 (s, 3H), 2.91 (s, 2H), 3.22 (m, 2H), 4.17 (t, J 7 Hz, 1H), 4.32 (m, 1H), 4.37 (m, 1H), 4.59 (br d, J 4.5 Hz, 2H), 5.21 (d, J 10.5 Hz, 1H), 5.30 (d, J 17 Hz, 1H), 5.83 (m, 1H), 5.88 (m, 1H), 6.26 (br s, 1H), 6.35 (br s, 2H), 7.26 (t, J 7.5 Hz, 2H), 7.37 (t, J 7.5 Hz, 2H), 7.57 (m, 2H), 7.74 (d, J 7.5 Hz, 2H).

¹³CNMR (CDCl₃, 125 MHz): □ 12.68, 18.22, 19.54, 25.69, 28.78, 29.93, 40.96, 43.43, 47.36, 53.72, 54.10, 66.23, 67.39, 86.63, 117.78, 119.12, 120.19, 124.93, 125.40, 127.34, 127.96, 131.79, 132.47, 133.17, 138.54, 141.49, 143.97, 144.08, 156.63, 159.03, 171.42). MS: (positive ESI) [M+H]⁺ m/z 689.

c) Allyl-N5-[[(4-ethyl-2,2,6,7-tetramethyl-2,3-dihydro-1-benzofuran-5-yl)amino](imino)methyl]-N²-[(4-nitrophenoxycarbonyl]ornithinate

Allyl-N²-[(9H-fluoren-9-ylmethoxy)carbonyl]-N⁵-{imino[(2,2,4,6,7-pentamethyl-2,3-dihydro-1-benzofuran-5-yl)amino]methyl}ornithinate (800 mg, 1.16 mmol) was dissolved in DMF (4 mL). Piperidine (1 mL) was added, and the reaction mixture was stirred at room temperature for 30 min and then concentrated. The resulting residue was dissolved in DCM (9 mL) and added to a suspension of 4-nitrophenylchloroformate (370 mg, 1.85 mmol) and pyridine (750 uL, 9.3 μmol) in DCM (6 mL) with cooling in an ice-salt bath. After stirring for 2.5 h, 1M KHSO₄ (20 mL) was added, the organic layer separated and the aqueous phase extracted with DCM (4×20 mL). The combined organic extracts were dried (MgSO₄), filtered, concentrated and the resulting residue purified by flash chromatography on silica gel (100% Hexane to 7:3 EtOAc:hexane) to afford allyl-N⁵-[[(4-ethyl-2,2,6,7-tetramethyl-2,3-dihydro-1-benzofuran-5-yl)amino](imino)methyl]-N²-[(4-nitrophenoxy)carbonyl]ornithinate (138 mg, 18%).

¹HNMR (CDCl₃, 500 MHz): □ 1.42 (s, 6H), 1.62 (m, 2H), 1.79 (m, 1H), 1.89 (m, 1H), 2.04 (s, 3H), 2.48 (s, 3H), 2.55 (s, 3H), 2.90 (s, 2H), 3.20 (m, 2H), 4.30 (m, 1H), 4.60 (br d, J 4.5 Hz, 2H), 5.22 (d, J 10.5 Hz, 1H), 5.29 (d, J 17 Hz, 1H), 5.86 (m, 1H), 6.25 (br s, 1H), 6.33 (br s, 1H), 6.50 (br d, J 6.5 Hz, 1H), 6.90 (d, J 7.5 Hz, 1H), 7.25 (d, J 8 Hz, 2H), 8.05 (d, J 7.5 Hz, 1H), 8.15 (d, J 8 Hz, 2H).

¹³CNMR (CDCl₃, 125 MHz): □ 12.63, 18.16, 19.45, 25.74, 28.76, 29.44, 40.8, 43.41, 54.41, 66.39, 86.71, 115.99, 117.78, 119.21, 122.22, 124.97, 125.23, 126.22, 131.66, 132.40, 133.02, 138.43, 140.75, 144.97, 153.45, 156.06, 156.67, 159.04, 163.07, 163.80, 171.6. MS: (positive ESI) [M+H]⁺ m/z 632.

d) Compound 1

Intermediate A (49.9 mg, 0.08 mmol) was dissolved in DMF (8 mL). Allyl-N⁵-[[(4-ethyl-2,2,6,7-tetramethyl-2,3-dihydro-1-benzofuran-5-yl)amino](imino)methyl]-A2-[(4-nitrophenoxy)carbonyl]ornithinate (60.6 mg, 0.096 mmol) was added, followed by DIEA (17 uL, 0.096 mmol) and the reaction mixture stirred at room temperature for 16 h. The reaction mixture was concentrated to give the crude urea. A solution of palladium(tetrakis)triphenylphosphine (8 mg, 0.0072 mmol) and dimedone (25 mg, 0.18 mmol) in TBF:DCM (1:1, 5 mL) was sparged with dry nitrogen and then added via canula to the urea and stirred at room temperature overnight to afford the crude carboxylic acid. The carboxylic acid was dissolved in DCM (1 mL), and p-Cresol (340 μL) and TFA (250 μL) were added and the reaction mixture stirred at room temperature for 20 h to afford crude Compound 1. The reaction mixture was purified by reverse phase HPLC (YMC basic semi prep column, linear gradient 65% Water (1% TFA) 35% MeCN (1% TFA)→100% MeCN (1% TFA)) to afford Compound 1 (11.3 mg, 17%). NMR and MS data were found to be identical with an authentic sample.

### Alternative Synthesis of Compound 1

The Intermediate of formula A was also prepared by the following route.

a) Synthesis of Intermediate C

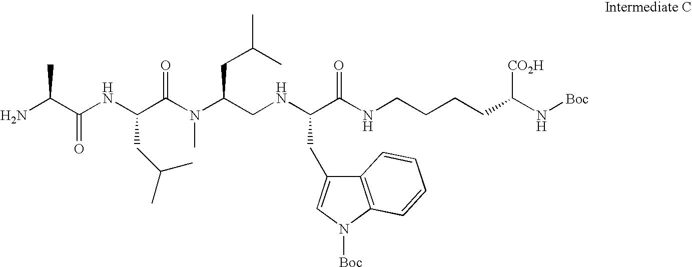

2-Chlorotrityl resin (300 mg, 0.42 mmol) was pre-swelled in DCM (2 mL) for 1 h. The resin was drained and a solution of Boc-D-Lysine(Fmoc)-OH (394 mg, 0.84 mmol) and DIEA (0.586 mL, 3.36 mmol) in DCM (2 mL) was added and the reaction mixture shaken for 1 h. A further aliquot of DIEA (0.293 mL, 1.68 mmol) was then added and the resin shaken for another 1 hr. Methanol (1 mL) was added to end-cap any unreacted resin and the reaction mixture shaken for a further 1 h. The resin was filtered and washed with DMF (2×5 mL), DCM (2×5 mL) and DMF (2×5 mL). The resin was then subjected to Fmoc-solid phase peptide synthesis (SPPS) using the following conditions:
(iii) Fmoc deprotection: 20% piperidine in DMF (4 mL) for 20 min followed by washing with DMF (4×5 mL), DCM (4×5 mL) and DMF (4×5 mL).
(iv) Coupling conditions: In all couplings a solution of the coupling reagent is added to the Fmoc-amino acid. This solution is added to the resin followed by DIEA. (a) Fmoc-Trp(Boc)-OH (0.885 g, 1.68 mmol), HBTU (0.5 M solution, 3.36 mL) and DIEA (0.293 mL, 1.68 mmol) 1 h. (b) Fmoc-N-Me-Leu-OH (0.617 g, 1.68 mmol), HBTU (0.5 M solution, 3.36 mL) and DIEA (0.293 mL, 1.68 mmol) 1 h. (c) Fmoc-Leu-OH (0.594 g, 1.68 mmol), HATU (0.5M, 0.639 g, 1.68 mmol in 3.36 mL DMF) and DIEA (0.293 mL, 1.68 mmol) 2 h. (d) Fmoc-Ala-OH (0.523 g, 1.68 mmol), HBTU (0.5 M solution, 3.36 mL) and DIEA (0.293 mL, 1.68 mmol) 1 h. Following all couplings the resin was filtered and washed with DMF (4×5 ml), DCM (4×5 mL) and DMF (4×5 mL). All couplings except for (c) were monitored using the ninhydrin test, coupling (c) was monitored using a bromophenol blue test.

Following Fmoc deprotection and thorough washing with DMF (4×5 ml), DCM (4×5 mL) and DMF (4×5 mL), the linear peptide was cleaved from resin with 2% TFA in DCM (150 mL) by rapid flow-wash into 250 mL of water. The DCM was removed in vacuo and the resulting solution frozen and freeze dried. The resulting gum was resuspended in 1:1 MeCN:H₂O (100 mL), frozen and freeze-dried to afford crude Intermediate C (265 mg, 0.276 mmol, 65.9% based on the 2-chlorotrityl resin).

b) Synthesis of Intermediate A

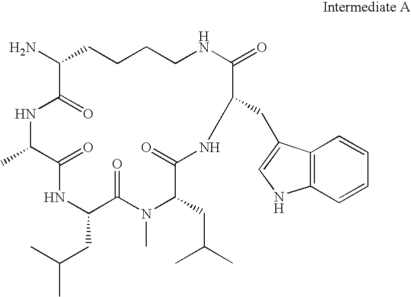

Crude Intermediate C (0.401 g, 0.419 mmol) and DIEA (0.438 mL, 1.26 mmol) in DMF (208 mL) were added dropwise with stirring to a solution of PyBOP (1.09 g, 2.10 mmol) and DIEA (0.146 mL, 0.838 mmol) in DMF (208 mL). The resulting solution was stirred at room temperature for 18 h then concentrated to dryness and partitioned between EtOAc (100 mL) and water (100 mL). The organic phase was washed several times with water (3×100 mL), dried (MgSO₄), filtered and concentrated to dryness. The crude product was treated with a solution of 90:9:1 (TFA:TIS_{[b1]}:DCM) for 2 h, concentrated to dryness and purified using reverse phase HPLC (95:5 H₂O (1% TFA):MeCN (1% TFA) to 3:2 H₂O (1% TFA):MeCN (1% TFA) over 60 min to afford Intermediate A (0.167 g, 0.226 mmol, 53.9%).

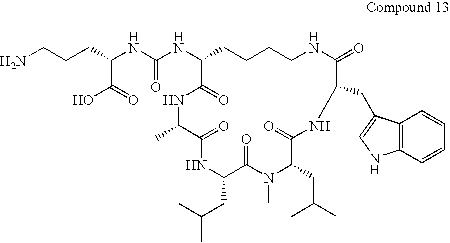

### Synthesis of Compound 13

Compound 13 was synthesised using a procedure similar to the procedure for Compound 1, starting from Intermediate A and N²-[(benzyloxy)carbonyl]-N⁵-(tert-butoxycarbonyl)ornithine. HRMS C₃₉H61N₉O₈ 822.4280 (M+H)⁺, found 822.4262.

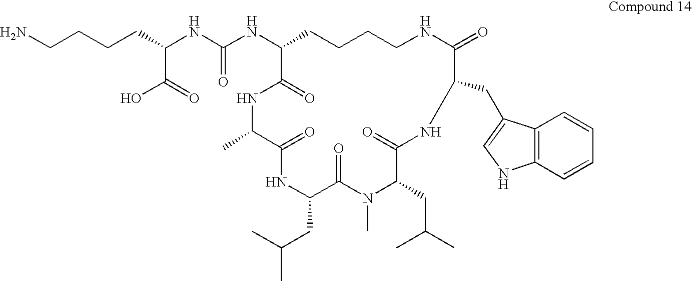

### Synthesis of Compound 14

Compound 14 was synthesised using a procedure similar to the procedure for Compound 1, starting from Intermediate A and tert-butyl N⁶-(tert-butoxycarbonyl)-L-lysinate.

¹H NMR (500 MHz, CD₃OD): □ 8.98 (d, 1H), 8.71 (d, 1H), 7.95 (dd, 1H), 7.79 (d, 1H), 7.64 (d, 1H), 7.31 (d, 1H), 7.08 (t, 1H), 7.01 (t, 1H), 6.78 (s, 1H), 5.00-4.88 (m, 2H), 4.78-4.70 (m, 1H), 4.36-4.23 (m, 2H), 4.19-4.13 (m, 1H), 3.88-3.77 (m, 1H), 3.55 (dd, 1H), 3.04-2.86 (m, 4H), 2.03-1.88 (m, 3H), 1.85 (s, 3H), 1.84-1.66 (m, 6H), 1.66-1.57 (m, 3H), 1.52 (d, 3H), 1.56-1.44 (m, 3H), 1.42-1.30 (m, 3H), 1.04 (two d, 6H), 0.95 (two d, 6H). HRMS (ESI) calculated for C₄₀H₆₄N₉O₈ 798.4878 (M+H)⁺, found 798.4858.

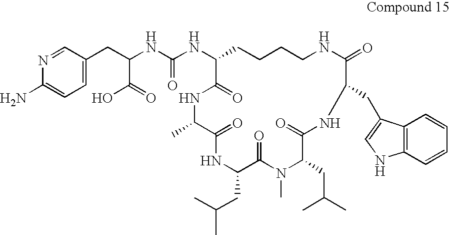

### Synthesis of Compound 15

Compound 15 was synthesised using a procedure similar to the procedure for Compound 1, starting from Intermediate A and 3-{6-[(tert-butoxycarbonyl)amino]pyridin-3-yl}alanine (WO 01/02364). HRMS C₄₂H₆₁N₁₀O₈ 833.4674 (M+H)⁺, found 833.4678.

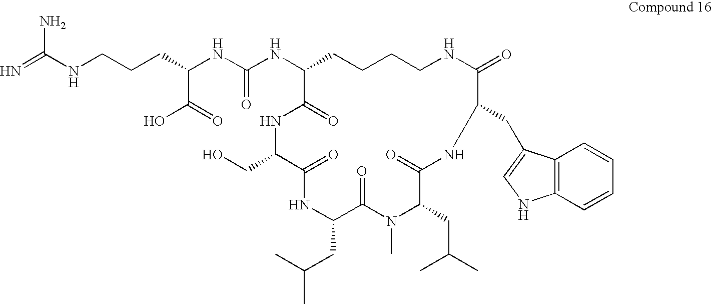

### Synthesis of Compound 16

a) Synthesis of Intermediate B

Intermediate B was synthesised using a procedure similar to the procedure for Intermediate A.

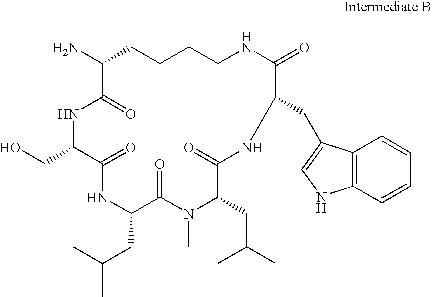

b) Synthesis of Compound 16

Compound 16 was synthesised according to the procedure for Compound 1, starting from Intermediate B.

¹H NMR (500 MHz, d₆-DMSO): □ 12.70 (broad s 1H), 10.83 (s, 1H), 8.86 (d, 1H), 8.47 (d, 1H), 7.70-7.79 (m, 3H), 7.57 (t, 1H), 7.46 (d, 1H), 7.45 (dd, 1H), 7.35 (d, 1H), 7.28 (d, 1H), 7.02 (dd, 1H), 6.96 (dd, 1H), 6.81 (broad s, 1H), 6.47 (d, 1H), 6.46 (d, 1H), 4.82 (m, 1H), 4.74-4.75 (ddd, 1H), 4.43 (ddd, 1H), 4.22-4.24 (m, 1H), 4.13 (ddd, 1H), 4.02 (ddd, 1H), 3.78 (dd, 1H), 3.71 (dd, 1H), 3.60 (m, 1H), 3.35 (m, 1H), 3.11 (dt, 2H), 2.86-2.92 (m, 1H), 2.78-2.80 (m, 1H), 1.83 (s, 3H), 1.79-1.83 (m, 1H), 1.52-1.56 (m, 1H), 1.57-1.60 (m, 1H), 1.60-1.64 (m, 3H), 1.69-1.70 (m, 1H), 1.42-1.48 (m, 5H), 1.33-1.36 (m, 1H), 1.22-1.25 (m, 2H), 1.18-1.20 (m, 1H), 0.95 (d, 3H), 0.91 (d, 3H), 0.89 (d, 3H), 0.85 (d, 3H). HRMS C₄₀H₆₄N₁₁O₉ 842.4888 (M+H)⁺, found 842.4885.

### Alternative Synthesis of Compound 16

The Intermediate of formula B was also prepared by the following route.

### Synthesis of Intermediate D:_{[b2]}

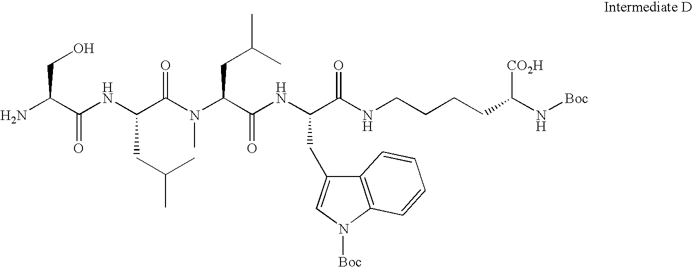

2-Chlorotrityl resin (1 g, 1.4 mmol) was pre-swelled in DCM (5 mL) for 1 h. The resin was drained and a solution of Boc-D-Lysine(Fmoc)-OH (1.31 g, 2.8 mmol) and DIEA (1.45 g, 1.98 mL, 11.2 mmol) in DCM (4 mL) was added and the reaction mixture shaken for 2 h. Methanol (1 mL) was added to end-cap any unreacted resin and the reaction mixture shaken for a further 1 h. The resin was filtered and washed with DMF (2×5 mL), DCM (2×5 mL) and DMF (2×5 mL). The resin was then subjected to Fmoc-solid phase peptide synthesis (SPPS) using the following conditions:
(i) Fmoc deprotection: 20% piperidine in DMF (4 mL) for 20 min followed by washing with DMF (4×5 mL), DCM (4×5 mL) and DMF (4×5mL).
(ii) Coupling conditions: In all couplings the solution of the coupling reagent in DMF is added to the Fmoc-amino acid. This solution is added to the resin followed by DIEA. (a) Fmoc-Trp(Boc)-OH (0.912 g, 1.732 mmol), HBTU (0.5 M solution, 3.46 mL) and DIEA (0.301 mL, 1.732 mmol) 1 h. (b) Fmoc-N-Me-Leu-OH (0.637 g, 1.732 mmol), HBTU (0.5 M solution, 3.46 mL) and DIEA (0.301 mL, 1.732 mmol) 1 h. (c) Fmoc-Leu-OH (0.612 g, 1.732 mmol), HATU (0.5M, 0.658 g, 1.732 mmol in 3.5 mL DMF) and DIEA (0.301 mL, 1.732 mmol) 2 h. (d) Fmoc-Ser(tBu)-OH (0.664 g, 1.732 mmol), HBTU (0.5 M solution, 3.46 mL) and DIEA (0.301 mL, 1.732 mmol) 1 h. Following all couplings the resin was filtered and washed with DMF (4×5 ml), DCM (4×5 mL) and DMF (4×5 mL). All couplings except for (c) were monitored using the ninhydrin test, coupling (c) was monitored using a bromophenol blue test.

Following Fmoc deprotection and thorough washing with DMF (4×5 ml), DCM (4×5 mL) and DMF (4×5 mL), the linear peptide was cleaved from resin with 2% TFA in DCM (400 mL) by rapid flow-wash into 500 mL of water. The DCM was removed in vacuo and the resulting solution frozen and freeze dried. The resulting gum was resuspended in 1:1 MeCN:H₂O (100 mL), frozen and freeze-dried to afford a crude Intermediate D (994.6 mg, 0.88 mmol, 63% based on the 2-chlorotrityl resin).

### Synthesis of Intermediate B:

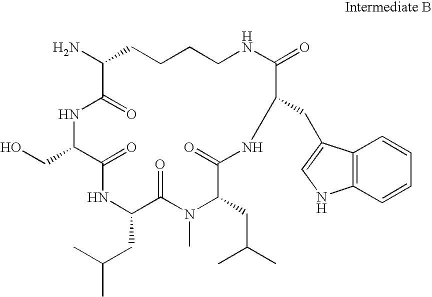

Crude Intermediate D (905 mg, 0.88 mmol) and DIEA (0.304 mL, 1.74 mmol) were dissolved in DMF (440 mL) and added dropwise with stirring to a solution of PyBOP (2.13 g, 4.1 mmol) and DIEA (0.918 mL, 5.3 mmol) in DMF (440 mL). Once addition was complete the resulting solution was stirred at room temperature for 20 h then concentrated to dryness to afford an orange gum, which was purified using Sephadex LH-20 (MeOH) to give the protected cyclic peptide (551 mg, 70%). The protected crude cyclic peptide was then treated with a solution of 95:2.5:2.5 (TFA:TIS:DCM) for 20 h. The reaction mixture was concentrated to dryness and purified using reverse phase HPLC (95:5 H₂O (1% TFA):MeCN (1% TFA) to 3:2 H₂O (1% TFA):MeCN (1% TFA) over 60 min to afford Intermediate B (214 mg, 32% from Intermediate D).

### EXAMPLE 5

The activities of certain Examples in the assay described in: Dirk Hendriks, Simon Sharpé and Marc van Sande, Clinical Chemistry, 31, 1936-1939 (1985), using a substrate concentration of 4 mM, are presented in Table I below.

TABLE I

| Compound No. | IC₅₀ |
|---|---|
| 2 | 0.1 μM |
| 8 | 2.5 μM |
| 12 | 0.2 μM |

### ABBREVIATIONS

EtOAc=ethyl acetate
DCCC=droplet counter current chromatography
MeOH=methanol
Leu=leucine
DMSO=dimethyl sulfoxide
Trp=tryptophan
HPLC=high pressure liquid chromatography
RPHPLC=reverse phase high pressure liquid chromatography
Boc=tert-butoxycarbonyl
Fmoc=(9H-fluoren-9-ylmethoxy)carbonyl
gHMBC=gradient heteronuclear multiple bond correlation
gCOSY=gradient correlated spectroscopy
gHSQC=gradient heteronuclear single quantum coherence
CPC=centrifugal partition chromatography
DIEA=diisopropyl ethyl amine
HATU=O-(7-Azabenzotriazol-1-yl)-N,N,N′,N′-tetramethyluronium hexafluorophosphate
HBTU=O-Benzotriazol-1-yl-N,N,N′,N′-tetramethyluronium hexafluorophosphate
THF=tetrahydrofuran
DMF=N,N-dimethylformamide
Lys=lysine
PyBOP=(benzotriazol-1-yloxy)tripyrrolidinophosphonium hexafluorophosphate
PyBrOP=bromo-tripyrrolidinophosphonium hexafluorophosphate
TIPS=Triisopropylsilane
TFA=trifluoroacetic acid
DCM=dichloromethane
MeCN=acetonitrile
Ala=alanine
Arg=Arginine
TIS=triisopropylsilane

1. A method for the treatment or prophylaxis of a disease or medical condition wherein inhibition of carboxypepsidase U is beneficial, said method comprising administering to a warm-blooded animal in need thereof an effective amount of a compound of formula (I): 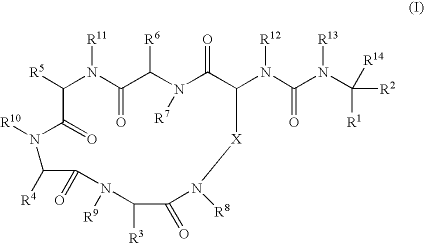
wherein:
X is (CH₂)ₘY(CH₂)ₙ;
m and n are, independently, 1, 2, 3, 4, 5 or 6; provided that m+n is not more than 6;
Y is a bond, O, S(O)ₚ, or S—S;
R¹ is CO₂R¹⁵ or a carboxylic acid isostere;
R², R³, R⁴, R⁵ and R⁶ are, independently, hydrogen, C₁₋₆ alkyl (optionally substituted by halogen, hydroxy, cyano, SH, S(O)₃H, S(O)_{q}(C₁₋₆ alkyl), OC(O)(C₁₋₄ alkyl), CF₃, C₁₋₄ alkoxy, OCF₃, COOH, CONH₂, CONH(C₁₋₆ alkyl), NH₂, CNH(NH₂), or NHCNH(NH₂)), C₃₋₆ cycloalkyl(C₁₋₄)alkyl (wherein the cycloalkyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)), heterocyclyl(C₁₋₄)alkyl (wherein the heterocyclyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)), phenyl(C₁₋₄)alkyl (wherein the phenyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)) or heteroaryl(C₁₋₄)alkyl (wherein the heteroaryl ring is optionally substituted by halogen, hydroxy, cyano, C₁₋₄ alkyl, CF₃, C₁₋₄ alkoxy, OCF₃, NH₂, CNH(NH₂) or NHCNH(NH₂));
p and q are, independently, 0, 1 or 2;
R⁷, R⁸, R⁹, R¹⁰, R¹¹, R¹² and R¹³ are, independently, H or C₁₋₄ alkyl;
R¹⁴ is H or C₁₋₄ alkyl; and,
R¹⁵ is H or C₁₋₄ alkyl;
or a pharmaceutically acceptable salt thereof.

2. A compound of formula (I): 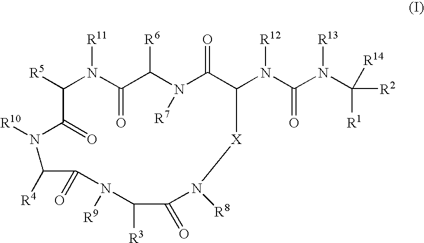
wherein:
X is (CH₂)₄;
R¹ is CO₂R¹⁵;
R² is C₁₋₆ alkyl, benzyl, straight-chain C₁₋₆ alkyl substituted at its terminus by NH₂, CNH(NH₂), NHCNH(NH₂) or (6-aminopyridin-3-yl)methyl; C₃₋₆ cycloalkyl substituted by NH₂, CNH(NH₂) or NHCNH(NH₂); heterocyclyl containing at least one nitrogen atom; non-nitrogen containing heterocyclyl substituted with NH₂, CNH(NH₂) or NHCNH(NH₂); heteroaryl substituted with NH₂, CNH(NH₂) or NHCNH(NH₂); phenyl substituted with NH₂, CNH(NH₂) or NHCNH(NH₂); heteroaryl(C₁₋₄)alkyl substituted with NH₂, CNH(NH₂) or NHCNH(NH₂); phenyl(C₁₋₄)alkyl substituted with NH₂, CNH(NH₂) or NHCNH(NH₂); or C₃₋₆ cycloalkyl(C₁₋₄)alkyl substituted with NH₂, CNH(NH₂) or NHCNH(NH₂); all of the above rings being optionally further substituted by one or more of: halogen, hydroxy, cyano, C₁₋₄ alkyl, CF₃, C₁₋₄ alkoxy or OCF₃;
one of R³, R⁴, R⁵ and R⁶ is independently, hydrogen, heteroaryl(C₁₋₄)alkyl (wherein the heteroaryl ring is optionally substituted by one or more of halogen, hydroxy, cyano, C₁₋₄ alkyl, CF₃, C₁₋₄ alkoxy, OCF₃, NH₂, CNH(NH₂) or NHCNH(NH₂)); and the others are, independently, hydrogen, C₁₋₆ alkyl (optionally substituted by halogen, hydroxy, cyano, SH, S(O)₃H, S(O)_{q}(C₁₋₆ alkyl), OC(O)(C₁₋₄ alkyl), CF₃, C₁₋₄ alkoxy, OCF₃, COOH, CONH₂, CONH(C₁₋₆ alkyl), NH₂, CNH(NH₂), or NHCNH(NH₂)), C₃₋₆ cycloalkyl(C₁₋₄)alkyl (wherein the cycloalkyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)), heterocyclyl(C₁₋₄)alkyl (wherein the heterocyclyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)), phenyl(C₁₋₄)alkyl (wherein the phenyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)) or heteroaryl(C₁₋₄)alkyl (wherein the heteroaryl ring is optionally substituted by halogen, hydroxy, cyano, C₁₋₄ alkyl, CF₃, C₁₋₄ alkoxy, OCF₃, NH₂, CNH(NH₂) or NHCNH(NH₂));
p and q are, independently, 0, 1 or 2;
R⁷, R⁸, R⁹, R¹⁰, R¹¹, R¹² and R¹³ are, independently, H or C₁₋₄ alkyl;
R¹⁴ is H or C₁₋₄ alkyl; and,
R¹⁵ is H or C₁₋₄ alkyl;
or a pharmaceutically acceptable salt thereof.

3. The compound of formula (I) as claimed in claim 2 wherein:
X is (CH₂)₄;
R¹ is CO₂R¹⁵;
R² is straight-chain C₁₋₆ alkyl substituted at its terminus by NH₂, CNH(NH₂) or NHCNH(NH₂); C₃₋₆ cycloalkyl substituted by NH₂, CNH(NH₂) or NHCNH(NH₂); heterocyclyl containing at least one nitrogen atom; non-nitrogen containing heterocyclyl substituted with NH₂, CNH(NH₂) or NHCNH(NH₂); heteroaryl substituted with NH₂, CNH(NH₂) or NHCNH(NH₂); phenyl substituted with NH₂, CNH(NH₂) or NHCNH(NH₂); heteroaryl(C₁₋₄)alkyl substituted with NH₂, CNH(NH₂) or NHCNH(NH₂); phenyl(C₁₋₄)alkyl substituted with NH₂, CNH(NH₂) or NHCNH(NH₂); or C₃₋₆ cycloalkyl(C₁₋₄)alkyl substituted with NH₂, CNH(NH₂) or NHCNH(NH₂); all of the above rings being optionally further substituted by one or more of: halogen, hydroxy, cyano, C₁₋₄ alkyl, CF₃, C₁₋₄ alkoxy or OCF₃;
one of R³, R⁴, R⁵ and R⁶ is independently, hydrogen, heteroaryl(C₁₋₄)alkyl (wherein the heteroaryl ring is optionally substituted by halogen, hydroxy, cyano, C₁₋₄ alkyl, CF₃, C₁₋₄ alkoxy, OCF₃, NH₂, CNH(NH₂) or NHCNH(NH₂)); and the others are, independently, hydrogen, C₁₋₆ alkyl (optionally substituted by halogen, hydroxy, cyano, SH, S(O)₃H, S(O)_{q}(C₁₋₆ alkyl), OC(O)(C₁₋₄ alkyl), CF₃, C₁₋₄ alkoxy, OCF₃, COOH, CONH₂, CONH(C₁₋₆ alkyl), NH₂, CNH(NH₂), or NHCNH(NH₂)), C₃₋₆ cycloalkyl(C₁₋₄)alkyl (wherein the cycloalkyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)), heterocyclyl(C₁₋₄)alkyl (wherein the heterocyclyl ring is optionally substituted by halogen, hydroxy, cyano, C₁₋₄alkyl, CF₃, C₁₋₄ alkoxy, OCF₃, NH₂, CNH(NH₂) or NHCNH(NH₂)), phenyl(C₁₋₄)alkyl (wherein the phenyl ring is optionally substituted by halogen, hydroxy, cyano, C₁₋₄ alkyl, CF₃, C₁₋₄ alkoxy, OCF₃, NH₂, CNH(NH₂) or NHCNH(NH₂)) or heteroaryl(C₁₋₄)alkyl (wherein the heteroaryl ring is optionally substituted by halogen, hydroxy, cyano, C₁₋₄ alkyl, CF₃, C₁₋₄ alkoxy, OCF₃, NH₂, CNH(NH₂) or NHCNH(NH₂));
p and q are, independently, 0, 1 or 2;
R⁷, R⁸, R⁹, R¹⁰, R¹¹, R¹² and R¹³ are, independently, H or C₁₋₄ alkyl;
R¹⁴ is H or C₁₋₄ alkyl; and,
R¹⁵ is H or C₁₋₄;
or a pharmaceutically acceptable salt thereof.

4. The compound of formula (I) as claimed in claim 2 wherein:
R¹ is CO₂R¹⁵;
R² is straight-chain C₁₋₆ alkyl substituted at its terminus by NH₂, CNH(NH₂) or NHCNH(NH₂); C₄ alkyl; or (aminopyridinyl)methyl;
one of R³ and R⁴ is (indol-3-yl)CH₂ optionally substituted by halo or hydroxy; and the other is benzyl (optionally substituted by halo or hydroxy) or C₄ alkyl;
or R³ and R⁴ are both methyl;
R⁵ and R⁶ are, independently, C₁₋₆ alkyl;
R⁷, R⁸, R⁹, R¹¹, R¹², R¹³ and R¹⁴ are H;
R¹⁰ is C₁₋₄ alkyl; and,
R¹⁵ is H or C₁₋₄ alkyl;
or a pharmaceutically acceptable salt thereof.

5. The method of claim 1 wherein X is (CH₂)₄.

6. The method of claim 1 wherein R¹ is CO₂R¹⁵ in which R¹⁵ is H or C₁₋₄ alkyl.

7. The compound as claimed in claim 2 wherein R² is straight-chain C₁₋₆ alkyl substituted at its terminus by NH₂, CNH(NH₂) or NHCNH(NH₂); C₄ alkyl; or (aminopyridinyl)methyl.

8. The compound as claimed in claim 2 wherein R² is C₁₋₆ alkyl, benzyl, or straight-chain C₁₋₆ alkyl substituted at its terminus by NH₂, CNH(NH₂), NHCNH(NH₂) or (6-aminopyridin-3-yl)methyl.

9. The compound as claimed in claim 2 wherein R² is straight-chain C₁₋₆ alkyl substituted at its terminus by NH₂, CNH(NH₂), NHCNH(NH₂) or (6-aminopyridin-3-yl)methyl.

10. The compound as claimed in claim 2 wherein R³ is CH₂indolyl, wherein the indolyl is optionally substituted by one or more of: halogen or hydroxy, C₁₋₄ alkyl or benzyl (optionally substituted by halogen or hydroxy).

11. The compound as claimed in claim 2 wherein R⁴ is CH₂indolyl, wherein the indolyl is optionally substituted by one or more of: halogen or hydroxy, C₁₋₆ alkyl or benzyl (optionally substituted by halogen or hydroxy).

12. The compound as claimed in claim 2 wherein R⁵ and R⁶ are, independently, C₁₋₆ alkyl.

13. The compound as claimed in claim 2 wherein R⁷, R⁸, R⁹, R¹¹, R¹², R¹³ and R¹⁴ are all H.

14. The compound as claimed in claim 2 wherein R¹⁰ is C₁₋₄ alkyl.

15. The compound as claimed in claim 2 which is a compound of the following formula 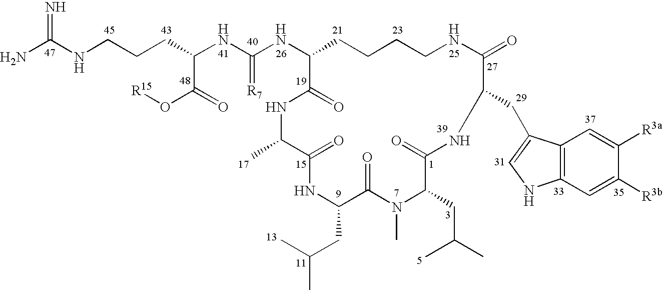
in which
R^{3a} is H, R^{3b} is H and R¹⁵ is H;
R^{3a} is OH, R^{3b} is Cl and R¹⁵ is H;
R^{3a} is OH, R^{3b} is Cl and R¹⁵ is CH₃;
R^{3a} is H, R^{3b} is H and R¹⁵ is CH₃;
R^{3a} is H, R^{3b} is Cl and R¹⁵ is H; 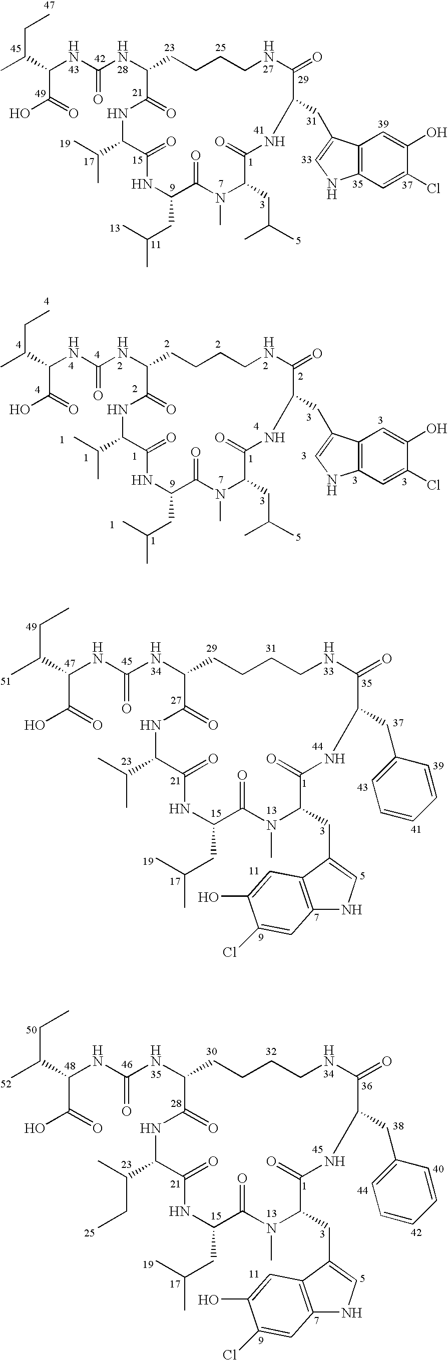 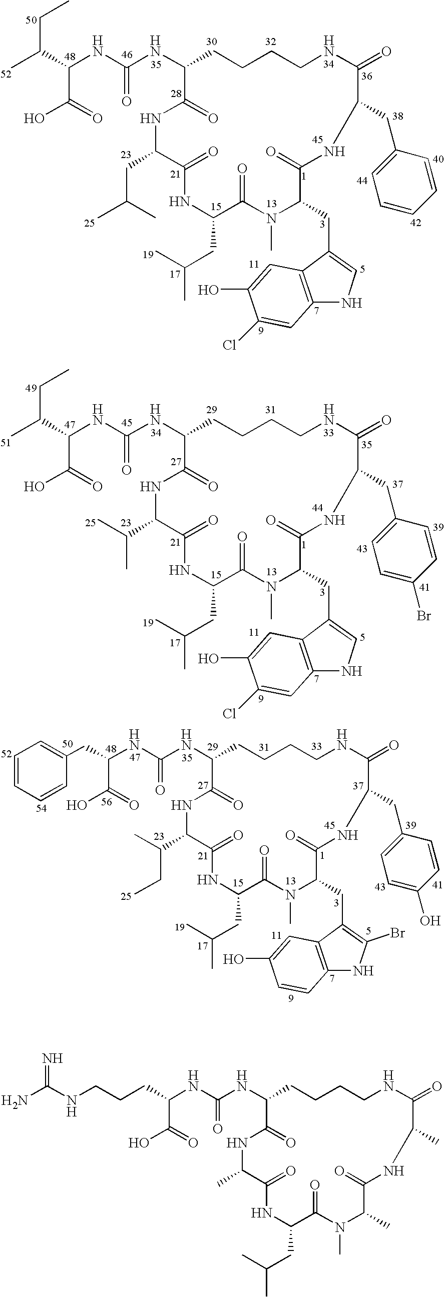 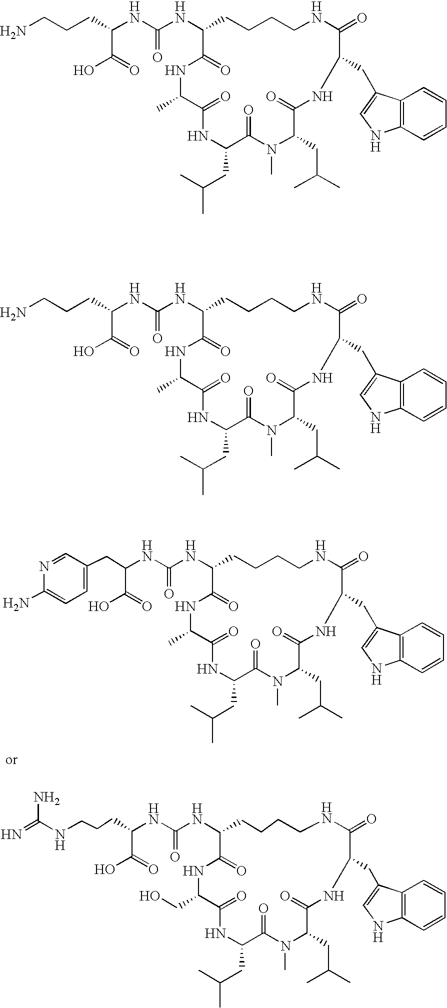
or a pharmaceutically acceptable salt thereof.

16. A method for the treatment or prophylaxis of a disease or medical condition wherein inhibition of carboxypepsidase U is beneficial, said method comprising administering to a warm-blooded animal in need thereof an effective amount of a compound of formula (I) or a pharmaceutically acceptable salt thereof as claimed in claim 2.

17. The method as claimed in claim 16 wherein said disease or medical condition is selected from thrombosis and/or hypercoagulability in blood and/or tissues; atherosclerosis; fibrotic conditions; inflammatory diseases; or a condition which benefits from maintaining or enhancing bradykinin levels in the body of a mammal.

18. A pharmaceutical formulation comprising a compound of formula (I) or a pharmaceutically acceptable salt thereof; as claimed in claim 2 as active ingredient in combination with a pharmaceutically acceptable adjuvant, diluent or carrier.

19. A compound of formula 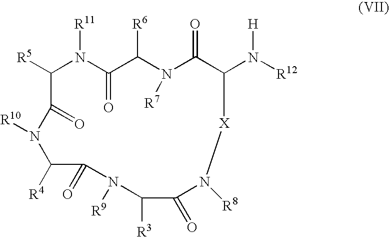
wherein R³ to R¹² and X are as defined in claim 2.

20. A process for preparing a compound as claimed in claim 19 which comprises treating a compound of formula VI 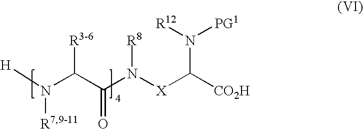
in which PG¹ is a suitable protecting group with a peptide coupling agent in the presence of a non-nucleophilic base in a polar aprotic solvent and then removing the protecting group.

21. A process for preparing a compound of formula I as claimed in claim 2 which comprises reacting a compound of formula VII as defined in claim 19 with a compound of formula VIII 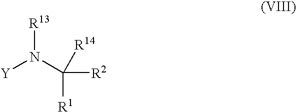
in which Y is an activated ester or NY is an isocyanate group.

22. The method as claimed in claim 1 wherein said disease or medical condition is selected from thrombosis and/or hypercoagulability in blood and/or tissues; atherosclerosis; fibrotic conditions; inflammatory diseases; or a condition which benefits from maintaining or enhancing bradykinin levels in the body of a mammal.
